(19) United States
(12) Patent Application Publication
(10) Pub. No.: US 2025/0295308 A1
(43) Pub. Date: Sep. 25, 2025

(54) OCULAR DISEASE MARKER IDENTIFICATION USING MULTI-SPECTRAL IMAGING

(71) Applicant: AI-SPECTRAL TECHNOLOGY CORP., Kanata (CA)

(72) Inventors: Nick Zeljko Ribaric, Ottawa (CA); Andrii Stepura, Toronto (CA); Kexing Liu, Nepean (CA)

(21) Appl. No.: 19/086,063

(22) Filed: Mar. 20, 2025

Related U.S. Application Data
(60) Provisional application No. 63/567,614, filed on Mar. 20, 2024.

(51) Int. Cl. A61B 3/12; A61B 3/00; A61B 5/00; G06T 7/00; G06T 7/194; G06T 7/33; G06T 7/60; G06T 7/73; G06V 10/143; G06V 10/25; G06V 10/764; G06V 10/82; G06V 40/18; G16H 10/60; G16H 30/40; G16H 50/20

(52) U.S. Cl. CPC A61B 3/12; A61B 3/0025; A61B 5/7264; G06T 7/0012; G06T 7/194; G06T 7/33; G06T 7/60; G06T 7/73; G06V 10/143; G06V 10/25; G06V 10/764; G06V 10/82; G06V 40/197; G16H 10/60; G16H 30/40; G16H 50/20; G06T 2207/10152; G06T 2207/20084; G06T 2207/30041; G06V 2201/03

(57) ABSTRACT

A method and system for the use of multi-spectral retinal images to achieve an effective, efficient, and AI enabled automated retinal disease biomarker detection using biomarker identification, segmentation, and quantification. A plurality of digital multi-spectral images of a retina of a patient at a plurality of illumination wavelengths can be done using a multi-spectral ophthalmoscope. The images can then be registered, processed, and assessed to automatically quantifying one or more biomarker based on the location and size of the biomarker in the plurality of processed multi-spectral images.

### CROSS-REFERENCE TO RELATED APPLICATIONS

This application claims priority to United States provisional patent application U.S. 63/567,614 filed on Mar. 20, 2024, which is hereby incorporated by reference herein in its entirety.

### FIELD OF THE INVENTION

The present invention pertains to a method for the use of multi-spectral retinal images to achieve an effective, efficient, and artificial intelligence (AI) enabled automated retinal disease biomarker detection.

### BACKGROUND

Retinal imaging techniques are used for early detection and diagnosis of ocular pathologies such as age-related macular degeneration (AMD), diabetic retinopathy (DR) and glaucoma. Retinal imaging techniques are also widely used to observe changes in retinal structure and to detect biomarkers. Conventional color fundus cameras use a white light source and collect an all-spectral combined color image. A multi-spectral retinal ophthalmoscope, in contrast, uses multiple individual illumination wavelengths across a wide wavelength range from visible to near infra-red (NIR). The multi-spectral retinal imaging (MSI) technique has proven to be an effective tool for enhanced visual identification of many of the above-mentioned biomarkers compared with conventional color fundus imaging.

Various clinical investigations have demonstrated the effectiveness of MSI for early detection and diagnosis of a variety of eye conditions and classification of retinal biomarkers in the process of disease detection and progression monitoring can be used together with a MSI technique. Generally, assessment by ophthalmologists with knowledge and experience is essential in diagnostic determination. These highly qualified retina experts are in high demand and frequently have a heavy workload.

Several methods have been proposed for automated biomarker segmentation in retinal imaging using color fundus images. The use of color fundus imaging is limited, however, in its clinical applicability at least due to limited tissue penetration of visible light frequencies, color sensor limitation, broad spectrum imaging, and the size of the datasets. In one example of retinal image analysis, U.S. Pat. No. 9,905,008 B2 to Katuwal et al. describes a method and system to automatically determine the side, field, and a level of image quality of fundus images of the retina of a human eye using image processing, computer vision and pattern recognition techniques to provide a process to identify and grade the quality of fundus images to improve efficiency and reduce errors in clinical and diagnostic retinal imaging workflows.

Automated pathology identification systems have the potential of accelerating retinal screening processes by alleviating the burden of manual lesion quantification for disease diagnosis and grading. In recent years, deep learning-based artificial intelligence (AI) approaches have achieved great successes in many areas of computer vision, surpassing traditional image processing techniques, and are now the de facto standard approach for most pathology detection tasks in retinal imagery. Deep learning approaches can make use of data overlooked or not observable by knowledge-based methods and have the potential of being easier to develop and/or train to provide automated methods of image analysis, diagnosis, and disease tracking.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide an automated retinal biomarker identification, extraction, and quantification from multi-spectral retinal images for diagnosis and tracking of retinal disease.

In an aspect there is provided a method of ocular biomarker identification comprising: obtaining a plurality of digital multi-spectral images of a retina of a patient at a plurality of illumination wavelengths using a multi-spectral ophthalmoscope; registering the plurality of multi-spectral images to provide a plurality of processed multi-spectral images that are scaled and aligned; identifying an ocular biomarker in at least one of the plurality of multi-spectral images; and automatically quantifying the biomarker based on the location and size of the biomarker in the plurality of processed multi-spectral images and the illumination wavelength at which the biomarker was identified.

In an embodiment, identifying the biomarker is based on the quantification of the biomarker at specific illumination wavelengths.

In another embodiment, the biomarker is one or more of a dry age-related macular degeneration (AMD) biomarker, diabetic retinopathy (DR) biomarker, geographic Atrophy (GA) biomarker, retinal tear, retinal detachment, hypertensive retinopathy, sickle cell retinopathy biomarker, epiretinal membrane (ERM) biomarker, central retinal vein occlusion (CRVO), branch retinal vein occlusion (BRVO), macular hole, retinitis pigmentosa biomarker, glaucoma biomarker, Stargardt disease biomarker, and cardiovascular biomarker.

In another embodiment, presence of the biomarker at a specific illumination wavelength enables differentiation of ocular biomarkers associated with specific ophthalmic diseases.

In another embodiment, registering the plurality of multi-spectral images comprises: identifying one or more retinal anchor and anatomical landmark; and defining retinal geographical coordinates.

In another embodiment, quantifying the biomarker comprises measurement of one or more of biomarker size, shape, density, intensity variations across spectral bands, and morphological characteristics.

In another embodiment, each of the plurality of multi-spectral image is a wide field of view image.

In another embodiment, the plurality of illumination wavelengths are selected based on the patient eye pigmentation.

In another embodiment, assessing quality of each of the plurality of processed multi-spectral images is done using a convolutional neural network based image qualification model.

In another embodiment, the plurality of multi-spectral digital images are obtained at illumination wavelengths about every 30-50 nm in the wavelength range of about 450 nm to about 940 nm.

In another embodiment, the plurality of illumination wavelengths includes autofluorescence wavelengths and infrared wavelengths.

In another embodiment, registering the plurality of multi-spectral images comprises one or more of denoising, artifact removal, geometric correction, contrast enhancement, and illumination equalization.

In another embodiment, registering the plurality of multi-spectral images comprises one or more of aligning the plurality of multi-spectral images to a common coordinate system, identifying a fovea center, and identifying an optic disk.

In another embodiment, each of the plurality of multi-spectral digital images is obtained by a multi-spectral ophthalmoscope in about 10 to 250 milliseconds, and the plurality of multi-spectral digital images are obtained by the multi-spectral ophthalmoscope in less than one second.

In another aspect there is provided a system for ocular biomarker identification comprising: a multi-spectral ophthalmoscope to capture a plurality of multi-spectral ocular images at a plurality of illumination wavelengths; an image registration module to pre-process and register the plurality of multi-spectral ocular images; a biomarker extraction module for isolating and quantifying an ocular biomarker in the plurality of multi-spectral ocular images, the biomarker extraction module comprising: a biomarker segmentation sub-module comprising a deep learning algorithm to differentiate relevant biomarkers from a background ocular structure; and a biomarker quantization sub-module.

In an embodiment, the biomarker extraction module further comprises a quality assessment module and region of interest (ROI) extraction module.

In another embodiment, the multi-spectral ophthalmoscope comprises an illumination system capable of ocular illumination at a plurality of illumination wavelengths in the range of about 450 nm and 940 nm.

In another embodiment, the biomarker quantization sub-module can provide a measurement of one or more of biomarker size, shape, density, intensity variations across spectral bands, and morphological characteristics of biomarkers in the plurality of multi-spectral ocular images.

In another embodiment, the biomarker extraction module is trained to identify an ocular biomarker selected from the group consisting of an dry age-related macular degeneration (AMD) biomarker, diabetic retinopathy (DR) biomarker, geographic Atrophy (GA) biomarker, retinal tear, retinal detachment, hypertensive retinopathy, sickle cell retinopathy biomarker, epiretinal membrane (ERM) biomarker, central retinal vein occlusion (CRVO), branch retinal vein occlusion (BRVO), macular hole, retinitis pigmentosa biomarker, glaucoma biomarker, Stargardt disease biomarker, and cardiovascular biomarker.

In another embodiment, the image registration module comprises a convolutional neural network to scale, align, and apply geographical coordinates to the plurality of multi-spectral images.

Embodiments of the present invention as recited herein may be combined in any combination or permutation.

### BRIEF DESCRIPTION OF THE FIGURES

For a better understanding of the present invention, as well as other aspects and further features thereof, reference is made to the following description which is to be used in conjunction with the accompanying figures which illustrate embodiments or aspects of the invention, where:

FIG. 1 illustrates a multi-spectral retinal image taken at different wavelengths;

FIG. 2 is a panel of multi-spectral retinal images;

FIG. 3 illustrates a method for training an AI model for retinal imaging diagnostic assessment;

FIG. 4 illustrates an automated drusen detection and quantification system for Age-Related Macular Degeneration (AMD);

FIG. 5 illustrates a quality assessment model for multi-spectral images for ocular biomarker identification;

FIG. 6 illustrates an optic disk segmentation module for ocular biomarker identification using multi-spectral imaging;

FIG. 7 illustrates a fovea center detection module for ocular biomarker identification using multi-spectral imaging;

FIG. 8 illustrates a drusen segmentation module for ocular biomarker identification using multi-spectral imaging.

FIG. 9 is a close-up view of the retina with individual drusen highlighted and an overlaid Early Treatment Diabetic Retinopathy Study (ETDRS) grid;

FIG. 10 is a bar chart identifying the occurrence and size of drusen in a retinal image;

FIG. 11 is a table indicating biomarker drusen quantification;

FIG. 12 provides a graph of a quality assessment model receiver operating characteristic (ROC) curve;

FIG. 13 illustrates quality assessment model qualitative results;

FIG. 14 illustrates a method for a continued AI learning algorithm in regional imaging;

FIG. 15 illustrates a method for automated ocular disease biomarker identification using multi-spectral imaging;

FIG. 16 illustrates a method for training an AI in ocular disease marker identification using multi-spectral imaging;

FIG. 17 illustrates a drusen segmentation model development workflow;

FIG. 18 illustrates a drusen candidate score calculation workflow;

FIG. 19 provides qualitative results in a drusen segmentation model;

FIG. 20 is an example of a set of multi-spectral retinal images with visible biomarkers;

FIG. 21 is a close-up retinal image at 660 nm showing drusen;

FIG. 22A is an example of an MSI image that does not have identified biomarkers;

FIG. 22B is an example of an MSI image that has identified and highlighted biomarkers; and

FIG. 23 illustrates identification of biomarkers in multi-spectral retinal images.

### DETAILED DESCRIPTION OF THE INVENTION

Unless defined otherwise, all technical and scientific terms used herein have the same meaning as commonly understood by one of ordinary skill in the art to which this invention belongs. Working examples provided herein are considered to be non-limiting and merely for purposes of illustration.

As used in the specification and claims, the singular forms “a”, “an” and “the” include plural references unless the context clearly dictates otherwise.

The term “comprise” and any of its derivatives (e.g. comprises, comprising) as used in this specification is to be taken to be inclusive of features to which it refers, and is not meant to exclude the presence of any additional features unless otherwise stated or implied. The term “comprising” as used herein will also be understood to mean that the list following is non-exhaustive and may or may not include any other additional suitable items, for example one or more further feature(s), component(s) and/or element(s) as appropriate.

As used herein, the terms “having,” “including” and “containing,” and grammatical variations thereof, are inclusive or open-ended and do not exclude additional, unrecited elements and/or method steps, and that that the list following is non-exhaustive and may or may not include any other additional suitable items, for example one or more further feature(s), component(s) and/or element(s) as appropriate. A composition, device, article, system, use, process, or method described herein as comprising certain elements and/or steps may also, in certain embodiments consist essentially of those elements and/or steps, and in other embodiments consist of those elements and/or steps and additional elements and/or steps, whether or not these embodiments are specifically referred to.

As used herein, the term “about” refers to an approximately +/−10% variation from a given value. It is to be understood that such a variation is always included in any given value provided herein, whether or not it is specifically referred to. The recitation of ranges herein is intended to convey both the ranges and individual values falling within the ranges, to the same place value as the numerals used to denote the range, unless otherwise indicated herein.

The use of any examples or exemplary language, e.g. “such as”, “exemplary embodiment”, “illustrative embodiment” and “for example” is intended to illustrate or denote aspects, embodiments, variations, elements or features relating to the invention and not intended to limit the scope of the invention.

As used herein, the terms “connect” and “connected” refer to any direct or indirect physical association between elements or features of the present disclosure. Accordingly, these terms may be understood to denote elements or features that are partly or completely contained within one another, attached, coupled, disposed on, joined together, in communication with, operatively associated with, etc., even if there are other elements or features intervening between the elements or features described as being connected.

Herein is described a system and method using multi-spectral image collection techniques in combination with machine learning techniques for multi-spectral retinal image analysis to achieve an effective, efficient, and AI enabled automated retinal disease biomarker detection. The presently described rapid and efficient visualization and analysis of retinal biomarkers from different modalities in the process of disease detection and progression monitoring provides integration of multi-spectral imaging data with AI generated analytical maps to allow for efficient extraction and presentation of impacting biomarkers in the eye. In particular, the present system and method provides temporal segmentation and quantification to support analysis of key biomarkers associated with a range of retinal diseases, including but not limited to drusen, melanin pigmentation in Retinal Pigmented Epithelium (RPE), retinal autofluorescence, micro aneurisms (MA), blood, neovascularization, hemes, and other related biomarker features knows to skilled in the art and which may be related to different diseases and longitudinal disease progression

The present method can provide identification of eye disease and quantification of disease progression. Using multi-spectral imaging (MSI), the present biomarker identification method and system can be used for automated retinal biomarker identification, extraction, and quantification from multi-spectral retinal images. In particular, the present MSI biomarker identification method uses deep learning to achieve AI inference for automated retinal biomarker identification, extraction, and quantification from multi-spectral retinal images. The present system and method can be used for a range of ocular biomarkers identification, extraction, and quantification from multi-spectral retinal images, including but not limited to biomarkers in ocular diseases such as Dry and Wet AMD, DR, glaucoma, retinal tumors, hypertensive retinopathy, central retinal vein occlusion (CRVO), branch retinal vein occlusion (BRVO), epiretinal membrane (ERM), and genetic diseases such as retinitis pigmentosa and Stargardt disease. In the present system and method a set of multi-spectral images of a retina is obtained, and from these images biomarkers can be identified in one or more of the images, and the biomarkers can be automatically quantified based on the location and size of the biomarker in the image.

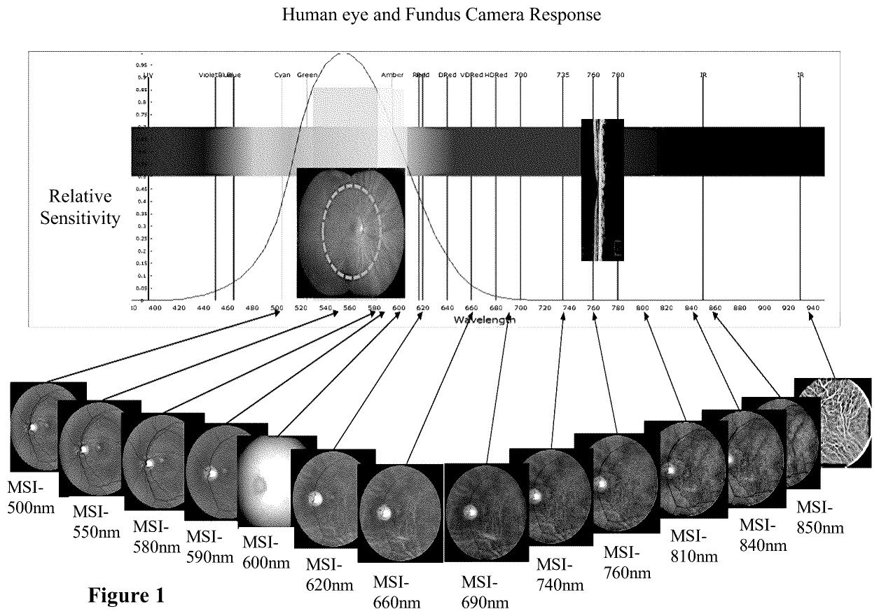

FIG. 1 illustrates a series of multi-spectral retinal images taken at different wavelengths. In multi-spectral imaging (MSI) of the eye, a series of images are captured substantially simultaneously which provide a plurality of images of the eye taken at different wavelengths. Using different wavelengths of light and capturing a plurality of images throughout the retina thickness allows for a visualization and quantification of different biomarkers that may be correlated to risk of eye disease progression. When compared to conventional fundus photography which uses a broad white light source and produces a limited spectrum of 480 nm to 600 nm, MSI offers a significantly wider spectral range with discrete spectral bands extending into the infrared region to 940 nm. This technique allows for observation of deep retinal structures including the Retinal Pigmented Epithelium (RPE) with pigmentary changes, choroid and sub-RPE drusen.

Multi-spectral images (MSI) of the retina and eye can be obtained on a multi-spectral imaging apparatus, also referred to as a multiwavelength ophthalmoscope or multi-spectral fundus camera. The MSI apparatus can be used by optometrists and ophthalmologists in a clinic to obtain a series of retinal images of a patient eye under test. Each of the multi-spectral images can be collected at a specific center wavelength, and preferably in a wide field of view, to provide a plurality of retinal images each taken at a specific wavelength. To obtain the plurality of multi-spectral images the retina is illuminated with a specific wavelength of light from at least one light source and then an image is captured at the specific wavelength by a digital imaging sensor such as a camera. This is in contrast to fundus retinal imaging or fundus photography, which generally uses white light, optionally with a color filter, to take a color image of the back of the eye, or fundus. In the case of multi-spectral imaging the camera or image sensor can be a monochromatic digital image sensor which senses luminosity or brightness rather than a color sensor since the fundus is being illuminated at a specific center wavelength of illumination light. In this way a monochromatic image can be captured for each of a plurality of different illumination wavelengths. In addition, fluorescence imaging can be done at a variety of wavelengths together with autofluorescence filters to image chromophores in the eye. Multi-spectral imaging can also be performed with the presence of dyes, for example fluorescein (IVFA) and indocyanine green (ICG) dye may be injected into patients blood stream to label specific retinal structures and allow imaging of normally invisible new retinal growth or changes, for example in the neovascular membrane.

The illumination light source used in MSI can be, for example, individual discrete light emitting diodes (LEDs), an array of LEDs, a hyper spectral laser, wide spectral tunable laser, or light transmitted from one of these sources through a fiber optic cable to light source assembly. Images can be captured through a set of lenses to form a quality image and the image can be received by a digital image sensor. In an embodiment, the image capturing by the imaging sensor can be synchronized with the illumination light source(s) by a trigger from a control module to maximize the efficiency of the imaging and reduce extra light exposure. Since the light direction of travel of the illumination light to the retina or fundus and the collected reflective image of the retina are in opposite directions, there are several methods that can be used to separate the illumination light from the reflected imaging light as much as possible to avoid direct reflection of illumination light back into the image sensor. In one control method, the imaging sensor can be set in a trigger mode, and illumination light source flash pulses are sent to the image sensor, where each light flash pulse will trigger the sensor to capture an image for the duration of the flash. In another control method the light source control is set in a trigger mode, and each time the image sensor is ready to capture an image the control system can send a trigger pulse to the light source control electronics, which in turn provides a control signal to flash the light source, one or multiple at a time, for a preset duration in concert with each image sensor capture.

The acquisition time to acquire a single image in multi-spectral imaging is typically less than 40 milliseconds, and for fundus autofluorescence the acquisition time is typically less than 250 milliseconds. Accordingly, the total time to provide a set of multi-spectral images for a single eye take less than a second. Preferably, the light illumination time for each image acquisition is between about 10 and 250 milliseconds. Short image acquisition times substantially minimizes any involuntary micro saccadic movements of the eye from blurring the image. The illumination light source flash duration time can be set for each illumination wavelength or illumination condition individually and for each flash. During focusing retinal image data is collected which allows calculation of the amount of energy required for imaging a specific retina pigmentary density. The darker the retina, the longer exposure is required, and this will allow the control module to proportionally adjust exposure for all wavelengths from short to near infrared (NIR). Accordingly, to obtain suitable multi-spectral images for each patient the present method can first determine a level of pigmentation of the patient's retina and adjust the illumination time for image capture for each of the plurality of images based on the level of pigmentation.

A multi-spectral retinal ophthalmoscope and multi-spectral fundus autofluorescence retinal imaging apparatus enables wavelength selection and spectral filtering to produce specific excitation or illumination light to illuminate the retina and to collect a corresponding retinal or fundus image including within the desired spectral range. For multi-spectral imaging, discrete wavelength illumination light can be generated and imaged across a wavelength range of, for example, 450 nm (blue) to 940 nm (near infrared). In fundus autofluorescence (FAF) the retina is illuminated with a specific wavelength and then an image is captured from the retinal with the correspondence fluorescence emission. Specifically, the fundus is illuminated with light of a first wavelength, and this light is absorbed by chromophores in the eye, which then re-emit light at a second different and longer wavelength. The image sensor or detector can then detect the re-emitted or fluorescence emission light from the retinal chromophores. Multi-spectral fundus autofluorescence can be achieved through the selection and paring of the excitation light source wavelength and the spectral filtering of a FAF filter. In some examples fluorescence autofluorescence imaging can provide FAF images with excitation wavelengths in blue (480 nm), green (550 nm), amber (600 nm), deep red (660 nm), and near-infrared (780 nm, 810 nm). The illumination or excitation light source for each specific wavelength FAF can be a series of individual discrete light sources such as light emitting diodes (LEDs) of the same spectral, individual lasers, a single hyper spectral laser, or a wide spectral tunable laser. Fluorescence images can be captured through the same set of lenses used in multi-spectral imaging together with an optical filter to screen out non-fluorescence light such as the excitation light or other stray light. One or more polarization filter can also be used during illumination and image capture for both multi-spectral imaging and FAF imaging. The combination of spectral filtering of illumination light and imaging light together with different spectral filters for illumination light and imaging light respectively with monochromatic digital imaging can provide high quality single wavelength as well as FAF images in a non-invasive, durable, and relatively inexpensive fundus imaging apparatus.

Shown are images taken at retinal imaging wavelengths from 475 nm (blue) to 940 nm-NIR, to progressively examine the different depth layers of the retina and choroid. Obtaining a retinal image every 30-50 nm in the wavelength range of about 450 to about 940 nm provides different image information for each wavelength since the depth of tissue imaged depends on the imaging wavelength used. In particular, longer wavelengths penetrate deeper into the structures of the eye and each image provides a different view of the retina depending on wavelength. Each monochromatic spectral slice represents successive images of the fundus as targeted and deliberately selected at different wavelengths. Each wavelength or monochromatic spectral slice differentially reflects, scatters, and absorbs deeper into the posterior pole and represents successive images of the fundus as targeted and deliberately selected at different wavelengths. Together, the set of multi-spectral images enhances differential visibility of the retinal and choroidal features at various tissue depths. Different illumination light wavelengths can thereby reveal different features within the retina that would be obscured by a white light image. For example, an image taken with an illumination light of 580 nm highlights oxygenated blood vessels and an image taken with an illumination light of 590 nm highlights de-oxygenated blood vessels. These images can also be combined using image processing to provide a clinically valuable map of retinal health. Images can be combined in multiple ways to arrive at a combined optical image with reduced specular reflection or other artifacts.

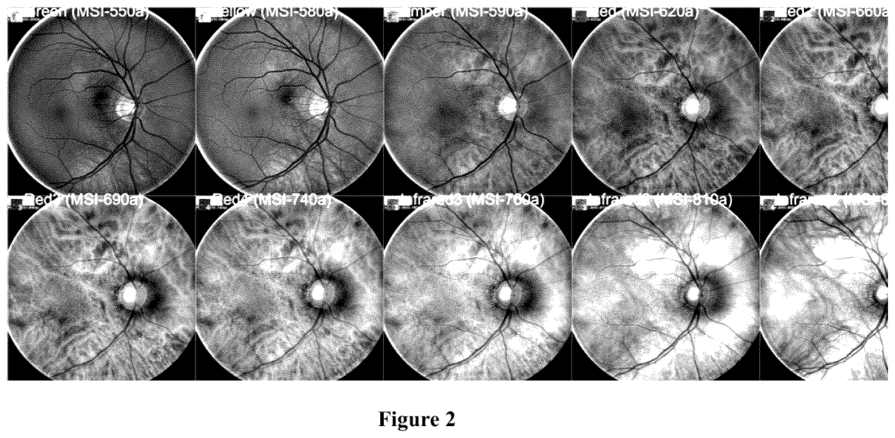

FIG. 2 is an example of a panel of pre-registered original multi-spectral retinal images. Obtaining multi-spectral retinal images enhances the visualization of the entire posterior pole of the eye. The pre-registration allows all images aligned and scaled to superimpose seamlessly so that precise analysis with location and size of any biomarkers identified can be reported accurately. The images are obtained at ten different wavelengths from the shortest wavelength of 550 nm on the top left to the longest wavelength of 850 nm to the bottom right, are highlighting different absorption of the retinal biological structures starting from the internal limiting membrane (ILM) through the retinal pigment epithelium (RPE) to the choroid.

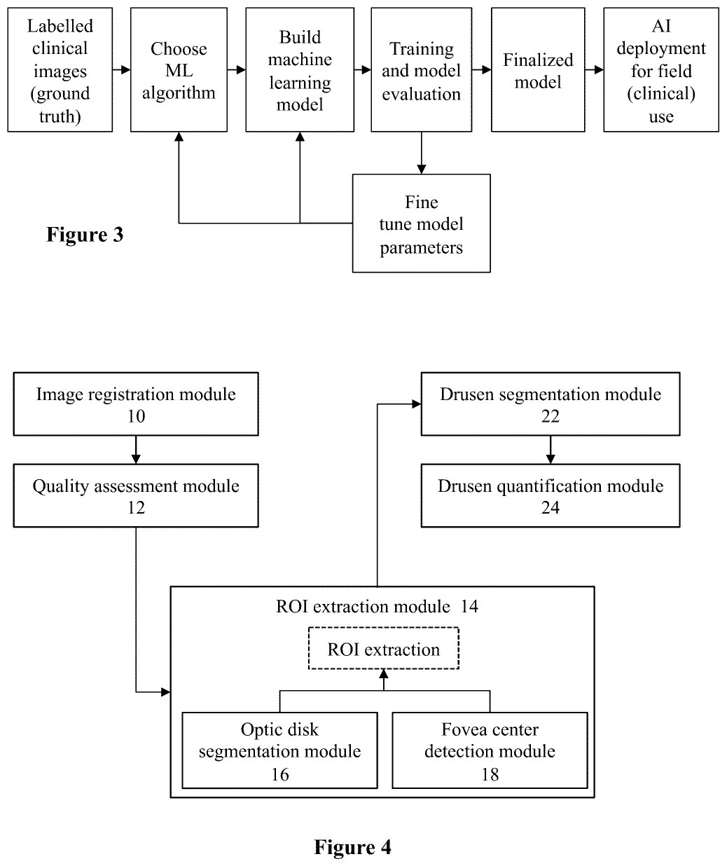

FIG. 3 illustrates a method for training an AI model for retinal imaging diagnostic assessment. For AI to be successfully utilized to assist disease screening, diagnostics, or progression monitoring in a clinical environment, an effective machine learning model must be developed, trained and validated properly. The first step in training an AI model for AI-assisted diagnostics in MSI is the collection of a large-scale dataset of MSI images. These images must be representative of a diverse patient population and cover a wide spectrum of eye conditions, retinal pathologies, anatomic features, etc. Cases of interest, such as examples of specific diseases are identified, reviewed and labeled by expert ophthalmologists and retinal specialists. These expert annotations provide the ground truth to enable supervised model training.

Following the collection and labeling of a dataset, the modeling workflow initiates with machine learning algorithm selection. Selecting the appropriate model architecture is a critical determinant of model performance. For the task of retinal image diagnostics, the selection process involves assessing deep learning methods such as convolutional neural networks (CNNs), vision transformers or other architectures, based on their ability to capture spatial and spectral features in MSI retinal images. Considerations include generalization capability, computational efficiency and performance in medical image processing benchmarks.

Once the appropriate machine learning (ML) architecture is chosen, a corresponding model is constructed using the framework of choice. The training process involves feeding the labeled dataset into the model to optimize the model parameters through a task-dependent loss function such as Dice loss for segmentation. Optimization techniques, such as stochastic gradient descent or more advanced methods in the like of Adam are applied to refine the model weights iteratively. Multiple iterations of model training or fine-tuning may be conducted until the key performance KPIs such as sensitivity, recall, F1-score and AUC-ROC are achieved on the hold-out test set. The refinement may involve picking a different machine learning algorithm, hyperparameter tuning, learning parameters adjustments and regularization. After the AI model passes extensive testing, it undergoes a production pipeline involving packaging, optimization and deployment in a clinical setting. The deployment strategies may involve containerized edge deployment on a GPU-accelerated MSI ophthalmoscope, as well as cloud deployment in the form of a web service.

An overview of an AI-based drusen segmentation and quantification system and method is described herein. It is understood that other biomarkers and applications that may be successfully addressed using this general model include but are not limited to: hemorrhage and microaneurysm (MA) segmentation and quantification, retinal vasculature segmentation and arterial-to-venular ratio (AVR) calculation, cup-to-disk (CDR) ratio calculation, and others.

FIG. 4 illustrates an automated drusen detection and quantification system for classification of Dry Age-Related Macular Degeneration (AMD). Dry AMD is a slow but progressive eye disease leading to atrophy of RPE and photoreceptors, leading to end stage vision loss due to Geographic Atrophy (GA). GA is a subtype of AMD and the number of people at risk from blindness from GA is ten times larger than from Wet AMD. Early detection and accurate assessment of Dry AMD is crucial for effective management and intervention. Among the key features associated with Dry AMD progression, drusen and drusen subtypes is of the highest importance. These lipid deposits accumulate beneath the retina and RPE and are considered early signs of the disease. Classification schemes such as that based on the Age-Related Eye Disease Study (AREDS) rely on parameters such as drusen size, number, and location from center of vision (macula) to assess the risk of Dry AMD disease progression. Herein, a method assisting AMD progression assessment is disclosed, which operates by automatically locating and quantifying drusen present in the image. Such assist-oriented approach greatly improves doctor's efficiency and accuracy, while also being highly interpretable.

The described method has the following core modules: image registration module 10; quality assessment module 12; region of interest (ROI) extraction module 14, drusen segmentation module 22; and drusen quantification module 24. The initial stage of the workflow involves the registration of a stack of MSI imaging data in image registration module 10. MSI images are obtained across multiple wavelengths in slightly different points in time, and due to factors such patient eye movements and optical misalignments, require registration (alignment) to establish a unified coordinate system. The registration process ensures pixel-wise correspondence across spectral bands necessary for further AI analysis. The quality assessment module 12 is responsible for evaluating the adequacy of MSI images before proceeding to biomarker identification stages. Retinal images may suffer from poor quality due to misalignment, defocus, patient noncompliance, or physiological factors such as small pupil size or cataracts, and as such require careful assessment on the subject of quality criteria correspondence. The module comprises of a convolutional neural network (CNN) trained on the diverse image quality dataset. The model assigns a quality score to each image in the stack, assessing whether it meets predefined acceptability thresholds. The quality score is then computed for the whole series to determine fit for further processing. By filtering out low-quality data, the model ensures that only diagnostically reliable data is processed in subsequent biomarker stages.

The region of interest (ROI) extraction module 14 determines retinal anchor points to define a standardized coordinate system for subsequent analysis. This module consists of two submodules: optic disk segmentation module 16; and fovea center detection module 18. The optic disk segmentation module 16 uses a fully convolutional neural network to segment the optic disk, while the fovea center detection module depends on a CNN to regress the fovea center coordinates. These biomarkers are then used to extract a ROI as defined by the Early Treatment Diabetic Retinopathy Study (ETDRS) grid for subsequent drusen segmentation and quantification.

The drusen segmentation module 22 applies a custom multi-encoder network to process the entire stack of MSI images, leveraging both local and global features as well as spatial dependencies between different spectral bands to achieve precise segmentation of individual drusen. This multi-scale and multispectral model approach enables robust differentiation of drusen from other retinal features. The output of the module is a segmentation mask which further undergoes quantification and statistical analysis. The drusen quantification module 24 computes key morphological and spatial parameters of drusen lesions based on the segmentation mask. After binarization and contour extraction, each detected drusen deposit is analyzed to determine its pixel area, minimum enclosing circle radius, and location within ETDRS-defined retinal zones. A statistical aggregation of drusen parameters is performed within each ETDRS region to derive clinically relevant indicators. The quantification follows the AREDS grading scheme, enabling automated assessment of disease severity.

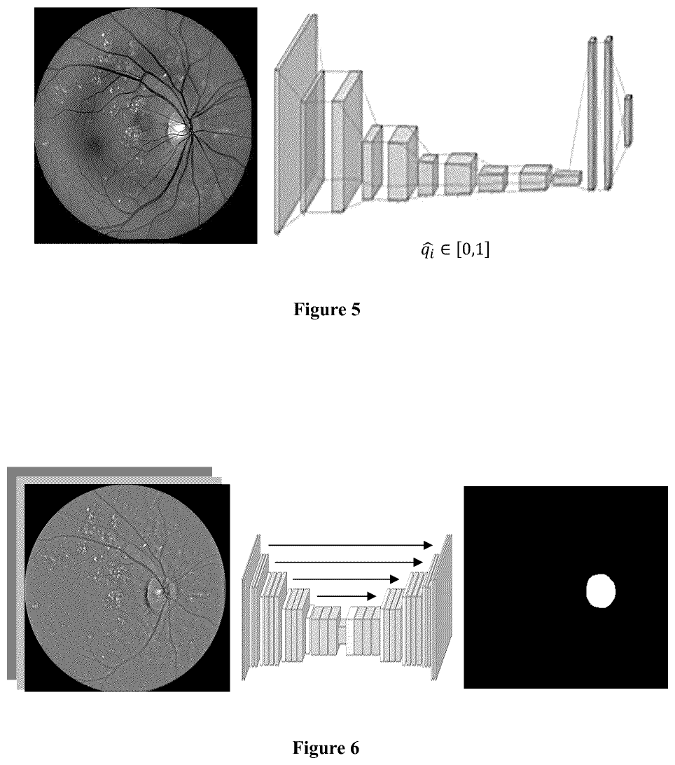

FIG. 5 illustrates a quality assessment model for multi-spectral images for ocular biomarker identification in a drusen segmentation model. During retinal image acquisition with a multi-spectral ophthalmoscope, retinal images may end up being of a poor quality due to multiple factors such as, for example, lack of user training, misalignment issues, patient noncompliance, patient inability to focus, and various physiological limitations such as very small pupil, presence of cataracts, involuntary eye movements, etc. Although these factors may be difficult to control, the quality assessment module in the present system and method can provide the operator with immediate analysis and guidance if the MSI image set should be retaken. In combination with a clinic-based apparatus for obtaining multi-spectral retinal images, the quality assessment module can provide immediate feedback on whether the set of retinal images taken at various wavelengths is of sufficient quality for analysis. This quality-based filtering can also serve an important role in candidate case location workflows, allowing to cut the search space by discarding studies of insufficient quality. Another characteristic of the MSI image set from a patient is that the images from different wavelengths may be slightly shifted with respect to each other due to factors such as patient eye movement and chromatic distortions in optical system. To accommodate for this shift, registration can be done for the set of MSI images to establish a shared coordinate system, prior to feeding in the images to the biomarker-specific models operating on multiple wavelengths.

In an embodiment, the workflow commences with a quality assessment module that accepts registered or aligned MSI images, preferably with basic image size 2048×2048 or larger, from the imaging device. The module then proceeds to verify the presence of all images in the spectrum of interest, for example ranging from 550 nm to 740 nm for drusen analysis. If some of the wavelengths are missing, for example if the registration process was not successful, the quality assessment module can abort the workflow. Each registered image, downscaled to 512×512, is passed through a trained convolutional neural network that predicts a quality score [symbol] ϵ[0,1], with a probability of belonging to the “acceptable” class. A discrete pass/fail score for the entire series of images can then be calculated as:

$$( min ( , … , ) \geq {t}_{1} ) ⋀ ( \frac{1}{n} {\sum }_{i}^{n} \geq {t}_{2} )$$

where t₁ and t₂ are the customizable thresholds.

Depending on rigor setting, t₁ and t₂ can be set to higher or lower values. Such a voting mechanism ensures an accurate and stable assessment of the series of MSI images as a whole. If the final discrete quality score totals to 0, further execution of the workflow is terminated. Otherwise, the images are forwarded to region of interest (ROI) or biomarker extraction stage. This ensures that only sufficient quality studies are analyzed by biomarker models to provide accurate predictions. In one embodiment, the quality assessment network implements an EfficientNetV2 L-size convolutional architecture, modified to accept greyscale images with only one channel, specifically black/white or luminosity/brightness.

The purpose of the ROI extraction module is to identify a retinal anchor and define retinal geographical coordinates. As each human eye is different, this needs to be done for every retinal image. This is also referred to as a region of interest (ROI) orientation for biomarker mapping and is done so that the relative location of the biomarkers to be identified can be defined and referenced to.

The present system preferably follows the Age-Related Eye Disease Study (AREDS) severity grading scheme (D. M. Davis et al. “The Age-Related Eye Disease Study severity scale for age-related macular degeneration: AREDS Report No. 17,” Archives of Ophthalmology, vol. 123, no. 11, pp. 1484-1498, 2005.) under which the region of interest used for drusen quantification is bounded by the standard Early Treatment Diabetic Retinopathy Study (ETDRS) grid, the area within two optic disk diameters from the center of the macula.

FIG. 6 illustrates an optic disk segmentation module for ocular biomarker identification using multi-spectral imaging. To regress optic disk diameter and fovea center coordinates, two deep learning models were employed. In the illustrated optic disk segmentation module a U-Net++architecture with EfficientNetV2 L-size encoder was used to perform 2.5D binary semantic segmentation of the optic disk, given the stack of registered 550 nm (Green) and 620 nm (Red) wavelengths, both downscaled to 512×512. Preprocessing was done using Gaussian illumination equalization and Contrast Limited Adaptive Histogram Equalization (CLAHE), applied to each of the available wavelengths individually. During inference, binary thresholding was applied to the upscaled model output with a threshold of 0.5, followed by the calculation of minimum enclosing circle. If the minimum enclosing circle was not found, the module terminated the further processing. Otherwise, the diameter of the optic disk d was approximated as the diameter of the found circle.

The present model was trained on 3,000 mixed adapted color/MSI images resized to 512×512 resolution with validation and evaluation performed on equal-sized sets of 250 cases each. Both validation and evaluation contained a 1:1 class ratio between third-party and in-house cases. Rare conditions were purposefully overrepresented in control sets and oversampled during train set augmentation, which involved spatial and intensity transformations. Stratification was further applied during dataset splitting, conditional on available categorical and numerical variables. The neural network was trained to minimize the Dice (F1) loss:

$$L = \frac{1}{n} {\sum }_{i}^{n} ( 1 - \frac{2 T P}{2 T P + F P + F N} )$$

where TP, FP, FN represent the true positives, false positives and false negatives respectively.

Cross-validated optic disk segmentation model performance is given in Table 1.

TABLE 1
Optic disk segmentation model mean validation
set performance on 5-fold cross-validation

| Dice (F1) | Accuracy | Recall | Precision | Specificity |
|---|---|---|---|---|
| 0.959 | 0.9984 | 0.979 | 0.942 | 0.999 |

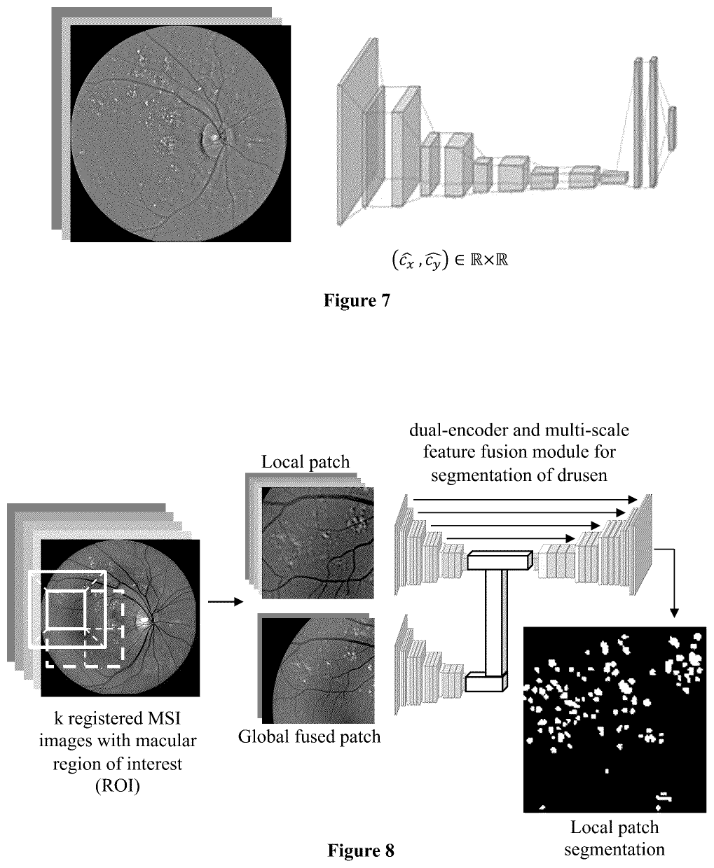

FIG. 7 illustrates fovea center detection module for ocular biomarker identification using multi-spectral imaging. A fovea center detection module follows an EfficientNetV2 L-size architecture, adapted to handle multiple channels to regress fovea center coordinates ( [symbol] , [symbol] ). Similar to optic disk segmentation module, fovea center detection accepts two registered 550 nm and 620 nm images, which are downscaled to 512×512 resolution with illumination equalization and CLAHE applied on top. During inference, a discretized model output was used as fovea center coordinates for subsequent calculations. The set of points falling withing the square region defined as

$$\{ ( x , y ) \vert x ∈ [ - 2 d , + 2 d ] , y ∈ [ - 2 d , + 2 d ] \}$$

were extracted from the image to form the ROI, with zero padding applied if necessary. If the overlap between the image space and the ROI was equal to 0, further execution of the workflow is terminated. Otherwise, the ROI was forwarded to drusen segmentation module.

To achieve uncompromised segmentation accuracy on full resolution MSI images while maintaining the global context, a custom dual-encoder modification of U-Net++ with EfficientNetV2 L-size encoder and multi-scale feature fusion module that accepts both local and global patches is used for the purpose of 2.5D binary semantic segmentation of drusen. Such scheme allows the model to produce high resolution segmentations of local patches, while utilizing the global context to correctly treat boundary regions and more accurately discern drusen from similar looking lesions according to the location in fundus image.

The fovea center detection module accepts a plurality of registered MSI images in original resolution with extracted ROI, ranging in illumination wavelength. In one embodiment, the MSI images are taken in the range of from 550 to 740 nm. The chosen spectrum range allows the identification of all known drusen subtypes, including subretinal drusenoid deposits as well as soft and cuticular drusen. To extract local patches, the images are split into n×n non-overlapping MSI patches of size 512×512 and 7 channels, with padding applied if necessary. The global patches were obtained by using overlapping 1024×1024 windows with the same stride used for the local patches. Furthermore, for the global patches, a single reduced-dimension fused image is obtained by averaging the seven wavelengths. The resulting local and global patches form a 1:1 correspondence, with both patches sharing the same center coordinates. Global patches were further downscaled to 512×512. During inference, the obtained segmentation masks for each of the patches are concatenated to arrive at the final segmentation mask. Gaussian smoothing was applied along stitching contours.

The model, based on a variant of EfficientNetV2 L-size architecture, was modified to accept two input channels and solve regression tasks, was trained to optimize for L1 loss:

$${L}_{1} = \frac{1}{n} {\sum }_{i}^{n} {\vert [ \begin{matrix}{c}_{x} \\ {c}_{y}\end{matrix} ] - [ \begin{matrix} \\ \end{matrix} ] \vert}_{1}$$

Cross-validation metrics for the fovea center detection model are provided in Table 2.

TABLE 2
Fovea center regression model mean validation
set performance using 5-fold cross-validation

| MAE (L1) | MSE (L2) | MAPE | R2 |
|---|---|---|---|
| 4.883 | 56.737 | 0.042 | 0.887 |

where MAE, MSE and MAPE stand for mean absolute error, mean squared error and mean absolute percentage error respectively.

FIG. 8 illustrates a drusen segmentation module for ocular biomarker identification using multi-spectral imaging. The image represents the inference result for the present drusen quantification model, which is a representative biomarker model. As an example of a well-trained submodule for qualitative biomarker identification, drusen inference is demonstrated. The original MSI image set is shown with overlaid data generated by deep learning with inferred segmented drusen from original data in comparison to expert labeled biomarker data. This provides an identification of represented and overlaid deep learning generated biomarker data. The module accepts k registered MSI images with macular region of interest extracted. Utilizing the entire stack of MSI images as an input to the convolutional neural network allows to learn deep spatial dependencies, ultimately providing enhanced discrimination of retinal structures.

The MSI stack is partitioned into local patches and fused global patches, leveraging the spectral richness of MSI to maximize segmentation accuracy. In this example, local patches (512×512) retain high-resolution spatial detail necessary for detecting small drusen deposits, while global fused patches (1024×1024 pixels, down sampled to 512×512) integrate broader contextual information. The global patches are obtained by averaging spectral bands to create a composite image that enhances structural contrast while maintaining spatial coherence. Such multi-scale approach, combined with MSI input data, ensures that small-scale lesion details are preserved while providing contextual awareness for accurate boundary delineation. Each local and global patch pair undergoes independent drusen segmentation, producing preliminary patch-level segmentation masks that highlight drusen deposits with spectral-based differentiation. The individual patch masks are then fused across the entire ROI, ensuring seamless integration of local per-patch segmentations into the ROI-wide mask. Refinement procedures including Gaussian smoothing at stitching boundaries further correct discontinuities and enhance segmentation accuracy.

The dual-encoder network architecture incorporates a multi-scale feature fusion module to process and reconcile local rich MSI data with global contextual features. One encoder processes high-resolution local patches, capturing fine morphological details of drusen, while the second encoder ingests global fused patches, preserving anatomical context to mitigate segmentation artifacts at lesion borders. The feature fusion module ensures that spectral and spatial features from both encoders are harmonized, enabling the network to accurately detect drusen and distinguish them from other lesions. This multi-scale, multi-spectral approach significantly outperforms traditional single-image segmentation methods by leveraging unique properties of MSI. In one example of the present system, a biomarker quantification submodule can be utilized to discretize the resulting mask obtained by the segmentation module with 0.5 threshold and further process it with contour extraction algorithm to identify individual biomarkers. Detected contours can be further processed and weak links are separated. Pixel area and the minimum enclosing circle radius can then be computed for each of the specific biomarker contours, as well as the associated the Early Treatment Diabetic Retinopathy Study (ETDRS) region. The aggregate statistics are subsequently computed for each of the ETDRS regions to arrive at the indicators defined by AREDS grading scheme.

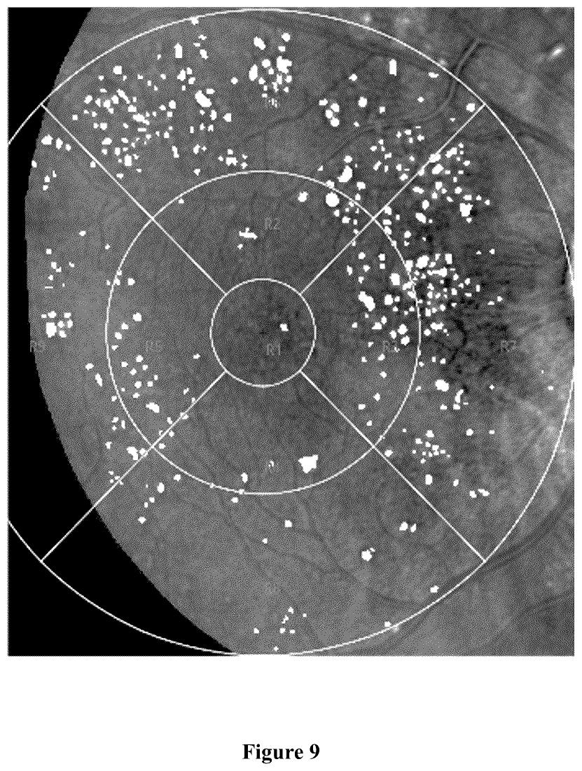

FIG. 9 is a close-up view of the retina with individual drusen highlighted and an overlaid Early Treatment Diabetic Retinopathy Study (ETDRS) grid. The MSI image shown has identified and highlighted biomarkers. The image represents the inference result for the present drusen quantification model, which is a representative biomarker model. The image shown is from the original MSI image set showing overlaid data generated by deep learning with inferred segmented drusen from original data in comparison to expert labeled biomarker data. This provides an identification of represented and overlaid deep learning generated biomarker data. As an example of a well-trained submodule for qualitative biomarker identification, drusen inference is demonstrated.

The aggregate drusen statistics are subsequently computed for each of the ETDRS regions to arrive at the indicators defined by AREDS grading scheme. The resulting deep learning analysis of all MSI wavelengths results in a map of biomarker presence and type at multiple wavelengths. The present method can identify that drusen are viewable in images present in a particular range of wavelengths at a particular location on the macula. In this case drusen can be automatically identified, quantified, and registered resulting drusen data is overlaid on top of the original image data to assist in rapid diagnosis. The computed statistics, as well as prediction masks and image overlays can be presented in a graphical user interface or viewer.

FIG. 10 is a bar chart identifying the occurrence and size of drusen in a retinal image displaying aggregate drusen statistics. The chart uses the bins set forth by AREDS scheme to show the distribution of drusen according to their sizes, color-coding different categories with respect to the severity they possess in the grading scheme.

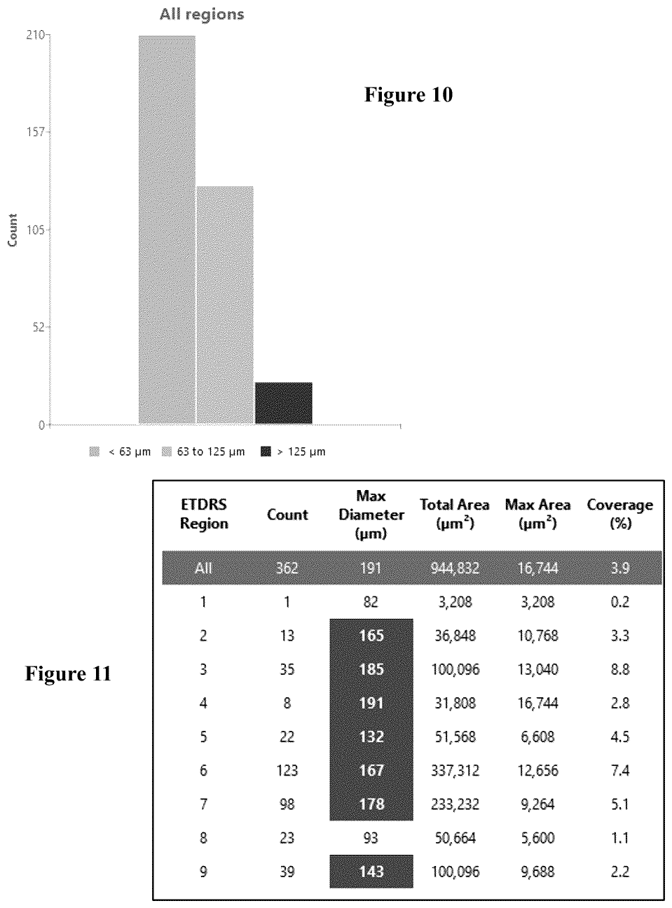

FIG. 11 is a table indicating biomarker drusen quantification. To enable the collection and utilization of the extensive number of diverse cases needed for the purpose of training the models which form the backbone of the described system, a carefully crafted data collection, labeling and model training workflow was designed for each of the modules. The quality assessment model development workflow has two stages, iterated until performance and data coverage targets were met: informed case sampling and labeling, performed based on prior knowledge or model output (either quality assessment model or biomarker-specific models); and training and validating the model on latest labeled data. To collect the initial data sample set of 5,000 patient eye cases, prior information on the presence of unsatisfactory quality cases such as studies performed in the lab during instrument trials was used to purposefully oversample such datapoints in light of anticipated class imbalance. Furthermore, to ensure representability of the composed sample, an unsupervised learning-based approach was used to find diverse cases among over 100,000 patient studies with multiple visits over 10 years period collected from clinics across North America. Latent space vectors obtained from the autoencoder trained on sampled clinic data were passed into the isolation forest to detect outliers. Computed embeddings were also used to form clusters which were subsequently sampled to form the core of the initial dataset. Lastly, categorical and numerical variables such as image wavelength, clinic, patient information were used to balance out the final sample distribution. Labeling was performed by the image acquisition expert under consultations with doctors on a two-pass basis: initial labeling and final approval.

During the second iteration, the quality dataset was expanded to 10,000 cases through the means of uncertainty sampling based on the predictions of the quality assessment model trained on the first iteration of the dataset, as well as further informed sampling guided by the lesion presence probabilities, as inferred by corresponding models. The technique of uncertainty sampling entailed that the cases on which the model prediction was close to 0.5 (i.e. the images which the model was not able to confidently classify into either “acceptable” or “unacceptable”) were more likely to be included in the new version of the dataset. In addition, scores computed by drusen and hemorrhage candidate search models were used to select cases suspected of containing moderate and severe pathologies in order to ensure the model exposure and correct handling of sick patients. Such an approach ensured that only the cases that were expected to contribute the most towards the improvement of the model's discrimination capabilities were used to expand the dataset, which maximized the size to value trade-off.

In the final iteration of the workflow, the quality assessment network based on L-sized EfficientNetV2 with one input channel was trained on 7,500 cases with 1,500 cases used for each of the validation and evaluation sets, with both sets containing an equal number of positive and negative examples (balanced). Stratification was used during splitting to ensure uniform coverage across clinics, conditions and other variables. The training set was further augmented by oversampling unsatisfactory quality images and applying affine transformation to synthesize new samples. The network was trained to minimize the weighted binary cross-entropy loss:

$$L = \frac{1}{n} {\sum }_{i}^{n} - α {q}_{i} log - ( 1 - {q}_{i} ) log ( 1 - )$$

where the weight for positive class α was set to 0.5 to match the train set class distribution. The quantitative and qualitative results obtained during quality assessment model validation are presented in Table 3.

TABLE 3
Quality assessment model mean validation set
performance on 5-fold cross-validation

| Accuracy | Recall | Precision | Specificity |
|---|---|---|---|
| 0.913 | 0.963 | 0.871 | 0.869 |

A classification scheme such as the AREDS can be used to diagnose and quantify parameters such as, for example, drusen size, number, and location, to assess disease state and/or progression. The presently described system and method is designed to be applicable in clinical workflows, with core modules trained and evaluated on numerous diverse cases obtained with MSI Imaging device The present approach to AMD progression assessment can be used to assist the specialists in the diagnostic process by automatically locating and quantifying drusen present in the image. Such assist-oriented approach greatly improves a clinician's efficiency and accuracy, while also being highly interpretable.

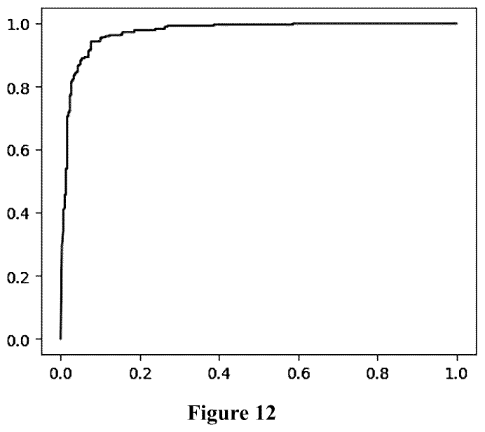

FIG. 12 provides a graph of a quality assessment model receiver operating characteristic (ROC) curve representing results of our quality assessment method.

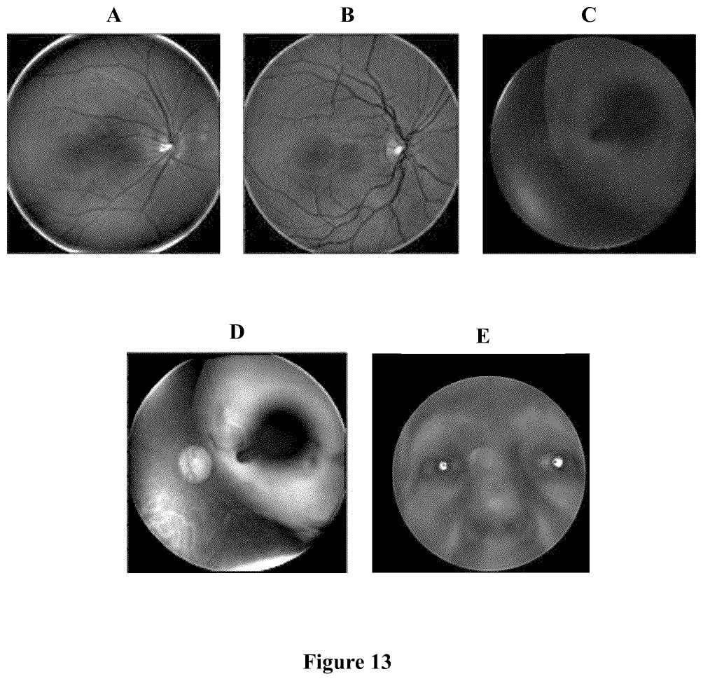

FIG. 13 illustrates quality assessment model with qualitative results. Pictured are the examples of correct model acceptance of high-quality scans (pictures A and B), rejection of low-quality scans (pictures C and D) as well as correct rejection of the out-of-distribution case (image of human face in picture E).

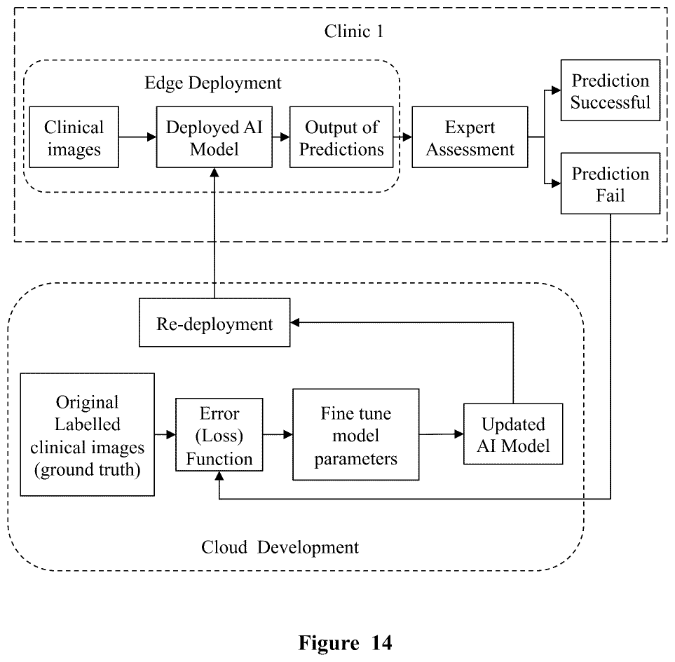

FIG. 14 illustrates a method for a continued AI learning algorithm in retinal imaging. In addition to an initial training model, a feedback path for clinical usage data, such as the diagnostic prediction error, can be fed back to the AI model to further fine-tune it, thus enabling continuous learning. The lower section of the diagram represents the AI training and development functional tasks as previously discussed. In this example these steps are performed in a cloud-based AI development environment. The upper section of the diagram represents the functions and flow of edge deployed MSI disease biomarker identification application in a clinical environment. Such an application environment, here shown as clinic 1, can be part of plurality of clinics, 2, 3, . . . n, each of which performs its own medical examination and collecting MSI retinal images of patients and can simultaneously perform AI operations to autodetect any or specific retinal disease biomarkers. The output of the AI prediction should be assessed by a medical expert or retinal specialist and any discrepancies by AI or errors of AI prediction can be fed back to the AI development center for retraining and refining the AI model. The updated model can then be scheduled to redeploy to each and every clinic. This constitutes a complete cycle of continued learning with distributed deployment sites.

In one example of an AI edge deployment in a location or a clinic, the AI module can be embedded and/or integrated in an MSI ophthalmoscope. Each set of MSI images for a patient that are captured can be automatically processed by the embedded AI module and the prediction results can become available almost immediately in the instrument or local MSI ophthalmoscope system environment. In another example of AI edge deployment, the MSI images captured by the MSI Ophthalmoscope can be stored in a hard disk, cloud, or other digital storage media, and analysed on an edge deployment AI module on a separate and independent computing device or on a network or cloud computer. The MSI images for the patient study can be retrieved from the digital storage and sent to one or more computing devices with deployed AI model for processing. The AI prediction results can then be stored in a pre-determined storage location and become available for review. An eye doctor or retinal specialist can assess the outcome of the AI analysis on a separate or independent computing device and provide expert judgment. In the majority of cases, AI models produce correct and accurate predictions, but in some cases the model may fail to provide the correct prediction. An expert confirmation may provide the correct identification and labeling of identified biomarkers on the collected images. The complete image data and correction information will then be sent to the AI development center to retrain and refine the parameters of the model.

The lower section of FIG. 14 representing the essentials of AI training and development for MSI retinal disease biomarker identification using multiple wavelength retinal images. In design and testing of the present system a significant number of previously captured clinical multispectral retinal images from a wide variety of patients were reviewed and labelled by retinal specialists identifying a specific disease marker; hence establishing the ground truth for training the AI model. After the training, the model was established and used for initial deployment. The complete and detailed description of the AI training and biomarker identification, segmentation, and quantization are described in more detail below.

The outlined image data fed by the failed AI prediction from the various clinics can be sent to the initial AI model trained with the ground truth. Feeding these prediction-failed images through an existing model will yield a higher error rate. These can be used to refine the AI model parameters in a reiterative training until such error rate is reduced to a pre-determined acceptable level. The AI model with refined parameters has much lower prediction error and can be packaged to an updated deployment module for a scheduled re-deployment to all clinics and edge devices, such as MSI ophthalmoscopes, that employ the model. This updating of the model completes a cycle of continued learning. The redeployment can be done, for example, through a prescheduled software upgrade. This learning cycle can be repeated and be continued on a regular basis for a continued learning algorithm.

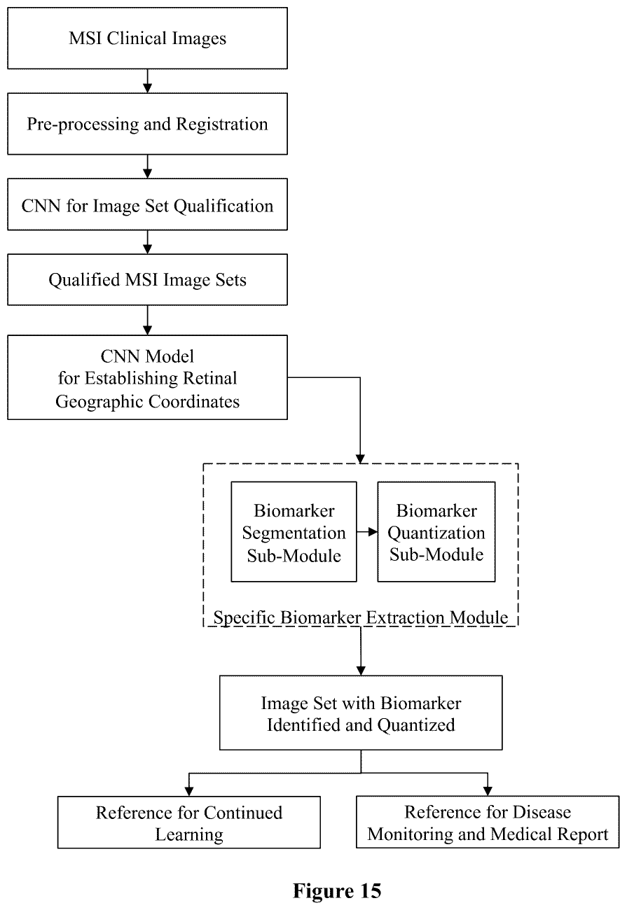

FIG. 15 illustrates an example method for automated ocular disease marker identification using multi-spectral imaging. The methodology disclosed herein uses conventional image processing techniques in combination with machine learning techniques with multi-spectral retinal images to achieve an effective, efficient, and AI enabled automated retinal disease biomarker detection and disease progression quantification which can be put into clinical usage. The process begins with the acquisition of multi-spectral imaging (MSI) clinical images. These images are obtained using MSI ophthalmoscopes that capture multiple spectral bands at various illumination wavelengths, allowing for enhanced differentiation of retinal biomarkers associated with various ophthalmic diseases. These MSI captures are essential to ensure accurate biomarker extraction and quantification.

Before analysis, the acquired MSI images undergo pre-processing and registration to standardize the data and enhance image quality. This step preferably includes denoising, artifact removal, geometric correction, contrast enhancement, illumination equalization, and other techniques. Processed series of MSI images are further registered (aligned) to a common coordinate system necessary for further AI processing. A CNN-based image qualification model is employed to assess the quality of each of the pre-processed and registered MSI images. This CNN model determines whether each image meets predefined criteria for clarity, contrast, feature visibility and others, effectively rejecting captures with poor visibility or focus, present artifacts or distortions, and other issues. Sets of MSI images that possess the sufficient aggregate quality score are deemed as qualified and proceed to the next stage. The CNN model is applied to the qualified MSI image sets to establish retinal geographic coordinates. This model maps key anatomical landmarks and regions of interest within the retinal structure, providing a spatial reference framework for biomarker extraction. Typically, the CNN-based localization system delineates key retinal features such as the fovea center and optic disk. By standardizing the spatial positioning of biomarkers, this stage ensures consistent localization across different patient datasets and follow-up assessments.

The specific biomarker extraction module is responsible for isolating and quantifying disease-specific retinal biomarkers and is comprised of two sub-modules: the specific biomarker segmentation sub-module and the specific biomarker quantization sub-module. The segmentation sub-module employs deep learning-based techniques to differentiate relevant biomarkers from the background retinal structure. The segmentation model is trained using pixel-wise annotations to achieve precise delineation of pathological features. Once segmentation is complete, the specific biomarker quantization sub-module performs extraction of individual features and subsequent numerical analysis on the extracted biomarkers. This may include the measurement of biomarker size, shape, density, intensity variations across spectral bands, and morphological characteristics. The quantization process ensures that the identified biomarkers are accurately characterized, providing objective, reproducible metrics that can be used for disease staging and risk assessment. The extracted biomarker data can serve as a reference for continued learning, contributing to the iterative improvement of the AI model. Ground truth annotations provided by expert ophthalmologists and retinal specialists can be incorporated into the training set to further refine the model's predictive capabilities. The final image set with identified biomarkers can further be utilized as a reference for disease monitoring and clinical reporting. The extracted biomarkers and associated statistics provide indicators that can be used to track disease progression over time. These data points can facilitate the assessment of treatment efficacy and support personalized therapeutic decision-making. Such AI-generated biomarker reports can serve as an essential tool for ophthalmologists, enabling precise and rapid data-driven decision-making in the management of retinal diseases.

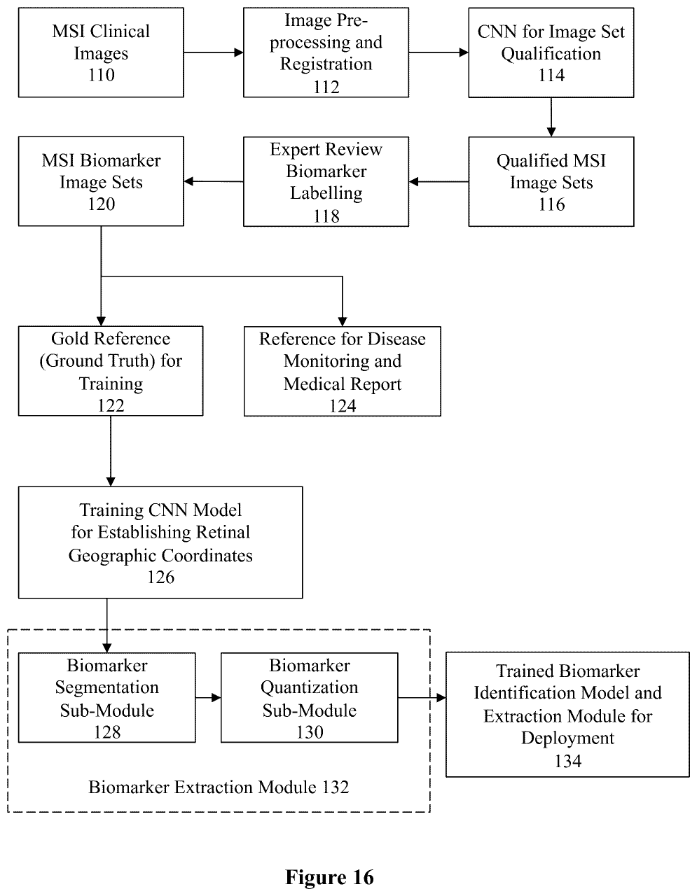

FIG. 16 illustrates a method for training an AI in ocular disease marker identification using multi-spectral imaging. Following the method described in FIG. 15, the workflow for training an AI in ocular disease marker identification commences with image processing, registration and quality assessment steps as described earlier. Specifically, MSI Clinical Images 110 are obtained and the images are pre-processed and registered 112. The MSI images are then sent to a CNN for image set qualification 114 to provide qualified MSI image sets 116. Expert review biomarker labelling 118 can then be done by expert ophthalmologists and retinal specialists manually review and annotate MSI biomarker image sets 120 to provide a gold-standard reference for model training and validation. This process involves identifying relevant retinal biomarkers, verifying their spatial locations, and confirming their clinical significance. The expert-labeled datasets serve as ground truth for supervised learning, ensuring that the model is trained on high-quality, expert-approved data.

The MSI biomarker image sets consist of qualified images that have undergone expert annotation to provide a gold reference or ground truth for training 122. These image sets contain both raw MSI data and the corresponding biomarker ground truth labels, forming the gold reference for model training and evaluation. The dataset is structured to include a diverse range of pathological cases, ensuring that the model generalizes well to new clinical scenarios. The annotated biomarker dataset serves as a reference for disease monitoring and medical reporting 124. The extracted biomarkers, along with their quantified metrics, allow for longitudinal tracking of disease progression.

The CNN model can then be trained using the gold reference dataset to establish precise retinal geographic coordinates 126. This training process involves mapping key anatomical structures, aligning biomarkers with specific retinal regions, and improving localization accuracy. By leveraging ground-truth labels, the CNN model learns to consistently identify and register retinal features across different patient datasets, enhancing the reliability of biomarker segmentation and quantification. The biomarker extraction module 132 undergoes extensive training to optimize its segmentation and quantization capabilities and can comprise a biomarker segmentation sub-module 128 and a biomarker quantization sub-module 130. The training process involves feeding the CNN with labeled biomarker datasets and iteratively adjusting model parameters to minimize segmentation errors. The module is refined using deep learning techniques, ensuring that it can accurately isolate and quantify retinal biomarkers from MSI data.

Upon completion of training and validation, the biomarker identification model and extraction module are finalized for deployment in clinical settings 134. The trained model can be integrated into diagnostic workflows, where it assists ophthalmologists in automated biomarker detection and disease assessment. The deployment-ready module is optimized for real-time inference and compatibility with medical imaging platforms, ensuring seamless integration into existing clinical infrastructure. The model's predictive outputs are used to generate automated reports, supporting enhanced decision-making in retinal disease management.

To enable AI inference leading to automated biomarker identification, extraction and quantization an AI model training or machine learning is utilized. The training method for ocular disease biomarker identification using multi-spectral imaging leverages a unique deep learning method to address the challenges on the extraction, classification, and identification of specific disease related biomarkers from the MSI images. Some biomarkers that can be detected using this method include but are not limited to hems, macular edema, fluid accumulation, blood leakage and deposits, drusen, exudates, micro aneurisms, cotton wool spots, retina atrophy, blood vessels change and tortuosity, atrophy of retinal pigment epithelium (RPE), and choroid atrophy.

The MSI image set contains wavelength specific information which relates to the depth and wavelength specific characteristics of the biomarker. For example, choroid related diseases occur deeper underneath the retinal RPE layer and therefore only show up in long wavelength such as infrared wavelength images. On the other hand, drusen, microaneurysms (MAs), cotton wool spots, exudates etc. reside in the retinal layers anterior to the outer segment layer, right below the inner limiting membrane of the retinal. These particular biomarkers are better viewed and reviewed with shorter wavelength images. In addition, biomarker features diminishing with longer wavelength can provide specific characteristics and indicia that can be used to identify a particular biomarker.

Provided is an example of training method for identifying and classifying biomarker drusen. Typical pre-registered original images are used and a neural network was trained to minimize the Dice loss (L) according to:

$$L = 1 - \frac{2 * T P}{2 * T P + F P + F N}$$

where
TP is the number of true positives,
FP is the number of false positives, and
FN is the number of false negatives.

MSI retinal images provide a rich collection of retinal information that is clearer and more accurate in presenting retinal information in a layered fashion. As MSI images are taken at different wavelengths and often at slightly separate times, the images need to be pre-processed to align and register. This is the pre-condition to perform image processing and deep learning for biomarker identification from a study of MSI images. A unique deep learning training method is described herein to qualify the MSI image set. Each MSI channel image is individually assessed and collectively qualified so the set of MSI images can be used for training, labelling, feature extraction, and quantification. This method can also rely on the quality score for all images taken at different wavelengths of the same subject eye.

The present method used to qualify a set of MSI images sends each image through a convolutional neural network that is trained to predict a quality score:
[symbol] ϵ[0,1]
   where the quality score indicates the probability of the image belonging to the “acceptable” class. A discrete pass/fail score for the entire series of images taken of the same subject can then determined as follows:

$$( min ( , … , ) \geq 0 \text{.5} ) ⋀ ( \frac{1}{n} {\sum }_{i}^{n} \geq 0.75 )$$

where [symbol] is the quality score for any image in the set of multi-spectral images.

This type of classification mechanism ensures an accurate and stable assessment of the series of images of the same subject. The MSI image set is qualified and can be used for image processing, manual annotation, further AI training, or AI inference of feature extraction and biomarker identification if the final quality score for the set of images is true or ≠0.

The qualified MSI image set can then be used for expert review, diagnosis, manual biomarker annotation, labelling, machine learning, AI training, or straight AI inference based on trained and deployed AI models. In addition to the conventional color fundus image review and annotation, the manual review of MSI image can use the present method to cross reference of all available images of different wavelengths to determine the types of the biomarker by characteristic association with known instances of the biomarker.

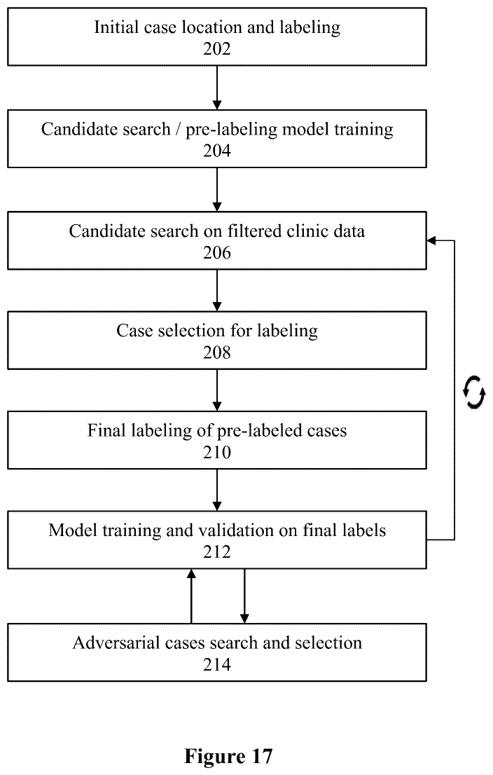

FIG. 17 illustrates a drusen segmentation model development workflow. The workflow commences with initial case location and labeling, which in turn enables to train candidate search and pre-labeling models to expedite the process of subsequent case location and annotation. The candidate cases undergo rigorous selection and labeling process with the assistance of AI-powered pre-labeling. The obtained train set is further enriched with adversarial cases for additional robustness. The model is then trained on the resulting dataset, which can be further expanded by repeating the aforementioned data collection steps.

Initially, a set of MSI data were obtained from a plurality of patients. For the initial labeled sample collection, known records of patients with AMD were used. Due to the purpose of the initial sample being the candidate search model training and subsequent compilation of a larger dataset, pseudo-labels were provided for each of the initial cases. Pseudo-labeling implied that not only drusen but visually similar features were also marked as positives with the intention of driving the resulting model sensitivity in light of limited initial training set size. In order to locate candidate cases likely to contain drusen, a ranking method guided by total segmentation area as produced by the associated model was opted for. The pixel area of the predicted mask was used to approximate the odds of encountering lesions of interest in the given case, with higher area corresponding to higher odds and vise-versa.

To enable fast inference on hundreds of thousands of available clinic studies, a segmentation model was designed to accept any single wavelength from the spectrum instead of the stack of MSI images. An ensemble method aggregating the model output for each of the individual wavelengths was used. The model, a U-Net++ with EfficientNetV2 L-size encoder, was trained on 45 512×512 images to perform binary segmentation of drusen candidates in the image. Elastic spatial and intensity transforms were applied dynamically during training and wavelengths were sampled uniformly from the spectrum of peak drusen visibility (590, 620 and 660 nm) to ensure correct operation on any single wavelength individually. The network was trained to minimize the focal loss as follows:

$$L = \frac{1}{n} {\sum }_{i}^{n} - α {{y}_{i} ( 1 - \hat{{y}_{ι}} )}^{γ} log \hat{{y}_{ι}} - ( 1 - α ) ( 1 - {y}_{i} ) {\hat{{y}_{ι}}}^{γ} log ( 1 - \hat{{y}_{ι}} )$$

where class weight α was set to 0.9 to strictly penalize false negatives. The focusing parameter γ was set to 2.

The same initial data sample was also used to train another model to perform pre-labeling of the subsequently located cases, where the predictions produced by the model were imported into the labeling tool as the suggested labels. The pre-labeling model used the same architecture and encoder as the candidate search model but differed in its input, which comprised of full MSI image stack rather than a single wavelength due to inference limitations being no longer applicable. The same optimization objective was used. To arrive at the final numeric value representing the drusen candidate score, the segmentation masks produced by the trained model when provided each of the selected MSI images were processed and each mask was binned with a window of size 50×50 to record the total number of pixels classified as drusen in each of the bins. Every combination of 1, 2 and 3 binned masks was matched to record a single overlap mask, where overlap is defined as the maximum number of pixels present in all of the masks for a particular bin. The consensus score for each of mask combinations considered is defined as the sum of the individual bin values in the resulting mask. Consensus scores for each of the number of masks considered are further averaged. Finally, the consensus scores were weighted and summed, with the most weight attributed to the score considering all three masks and the least weight—to the individual masks with no consensus scoring.

The complete function f for computing the candidate score ϵ[0, 512*512] was defined as:

$$f ( {M}_{590} , {M}_{620} , {M}_{660} ) = 0.1 * ( \frac{{M}_{590} + {M}_{620} + {M}_{660}}{3} ) +$$

$$+ * ( \frac{min ( {M}_{590} , {M}_{620} ) + min ( {M}_{590} , {M}_{660} ) + min ( {M}_{620} , {M}_{660} )}{3} ) +$$

$$+ 0.6 * sum ( min ( {M}_{590} , {M}_{620} , {M}_{660} ) )$$

where M₅₉₀, M₆₂₀ and M₆₆₀ are the binned segmentation mask matrices, min performs the minimum operation on each of the matrix entries and sum performs the summation of matrix entries.

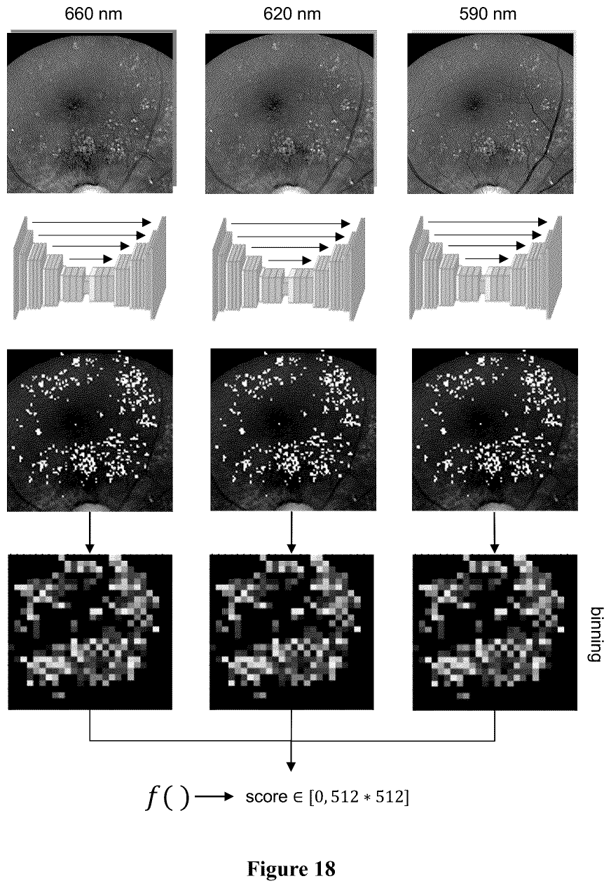

FIG. 18 illustrates a drusen candidate score calculation workflow. The candidate search procedure was applied on all clinic data satisfying the set quality thresholds, as predicted by the quality assessment model described earlier. The computed candidate scores were used to rank and select over 2,000 cases with the highest scores. These cases were subsequently forwarded to doctors for case selection and labeling. During the case selection process, two retinal specialists were presented with the candidate cases located and asked to indicate which of the cases were indeed of patients with AMD and characteristic drusen and which were either false positives or examples of other pathologies. Furthermore, for each of the true positive cases the doctors were asked to provide the approximate quantity of drusen (<10, <50, >50), their size and types, as well as the presence of other pathologies and condition severity.

The provided information was used to schedule cases for labeling, with early-stage AMD cases containing few or several drusen handled in the first labeling batches, and more severe cases postponed for later. This allowed the training of a pre-labeling model on simple cases which required less labeling time and predict labels for the more complicated cases with numerous drusen, significantly reducing the overall labeling time as the result. To date, over a thousand cases have been reviewed with 500 cases scheduled for labeling. Sampling was conducted in a way to ensure the uniform representation of different conditions including instances of soft, cuticular and sub-RPE drusen, patients with early, mild and severe AMD, as well as cases with other pathologies and diseases present such as geographical atrophy (GA), DR and ERM.

Cases scheduled for labeling were first processed by the “pre-labeling AI model” to predict suggested labels. These segmentation masks were further processed to extract individual polygons representing drusen to be imported into the labeling tool used by the doctors. Labeling was performed by a retinal specialist supervised by another senior expert in two stages: initial labeling and approval pass. The doctors followed technical guidelines established by the engineering team to ensure consistent and unbiased decision making. The process involved identifying drusen present in the image, verifying the correctness of the proposed label and adjusting the label by drawing and/or removing polygons around each of the individual lesions using specialized labeling software. Fused images obtained by averaging MSI images within the defined spectrum were used as labeling targets to ensure peak visibility of lesions at different depths. Doctors were referencing original MSI studies throughout the process as well. After each subsequent labeling batch, which comprised of around 50 cases, the re-training of the pre-labeling model was triggered. This ensured that the labels suggested to doctors were the most up-to-date and as accurate as possible. To date, 500 cases have been labeled and approved by the doctors.

To facilitate the development of advanced discriminatory capabilities in the final drusen segmentation model, the technique of adversarial training was employed. Such an approach entailed the enrichment of the dataset with cases containing visually similar but distinct pathologies and lesions. During the selection process, cases satisfying the criteria were recorded and subsequently added to the training dataset used for final model training. Masks for such cases were left empty to force the model to avoid classifying visually similar lesions as drusen. Adversarial conditions included exudates, epiretinal membrane, geographic atrophy, and others.

During model trials on broad clinic data, false positives were also recorded and selectively added as adversarial examples. This ensured that not only expert knowledge was used to guide model fine-tuning, but also the direct performance. Over 500 adversarial cases have be collected in total so far. The final composite dataset comprising of nearly a thousand true and adversarial cases was split into sets of sizes of 1000, 100 and 200 to serve as the training set, validation and evaluation sets respectively. Stratification was used to ensure uniform distribution of various conditions in all the sets.

For the purpose of model selection and hyperparameter tuning, a 5-fold cross-validation was performed on the original dataset. The experiments involved comparing different segmentation model architectures and encoders, optimization criterions and others. The top performing model, a previously described custom dual-encoder modification of the U-Net++architecture, was trained to minimize the Focal loss:

$$L = \frac{1}{n} {\sum }_{i}^{n} - α {{y}_{i} ( 1 - \hat{{y}_{ι}} )}^{γ} log \hat{{y}_{ι}} - ( 1 - α ) ( 1 - {y}_{i} ) {\hat{{y}_{ι}}}^{γ} log ( 1 - \hat{{y}_{ι}} )$$

Setting α to 0.75 and keeping γ at 2 has been empirically shown to yield the best overall Dice score while keeping the sensitivity nearly on par with the most sensitive models. The training of the final model was performed for 100 epochs using the AdamW optimizer, which is an improved version of the Adam optimizer that incorporates weight decay to reduce overfitting and improve generalization. The optimization process involved adjusting the model's parameters iteratively using mini-batch gradient descent, where the gradients were computed via backpropagation and used to update the model weights. The learning rate was adjusted dynamically using a cosine learning rate scheduler, which gradually reduced the learning rate following a cosine decay curve to promote smoother convergence. Additionally, early stopping was enabled to monitor validation performance and prevent overfitting. If the model's validation loss did not improve for a predefined number of epochs, training was halted early to avoid unnecessary computations and retain the best-performing model.

Data augmentation was applied dynamically during training, which included elastic and affine spatial and intensity transformations.

Current and possible KPI performance for drusen training is shown in Table 4.

TABLE 4
Drusen segmentation model mean validation set
performance using 5-fold cross-validation

| | Dice (F1) | Accuracy | Recall | Precision | Specificity |
|---|---|---|---|---|---|
| Current | 0.74 | 0.9983 | 0.77 | 0.7177 | 0.999 |
| KPI target | 0.80+ | 0.999 | 0.80+ | 0.80+ | 0.999 |

The output of the expert reviewed and annotated images can be used for medical reports and can also be used as part of the training set for AI deep learning or continued learning towards automated biomarker extraction. To automatically extract and present the biomarkers precisely, the system identifies a retinal anchor and defines the retinal geographical coordinates. This is also referred to as a region of interest (ROI) orientation for biomarker mapping and is done so that the relative location of the biomarkers to be identified can be defined and referenced to. This can be achieved through a trained and self-contained convolutional neural network (CNN). Following the generally accepted Age-Related Eye Disease Study (AREDS) severity grading scheme, the region of interest used for biomarker quantification can be bounded by the standard Early Treatment Diabetic Retinopathy Study (ETDRS) grid, which is the area within two optic disk diameters from the center of the macula. To do this, the optical disk is identified and the center of the fovea located so that these can be used as geographic reference.

For the present MSI image biomarker mapping, a U-Net++ architecture with EfficientNetV2 L-size encoder was used to perform 2.5D binary semantic segmentation of the optic disk. Similarly, an EfficientNetV2 architecture is adapted to handle multiple channels to regress fovea center coordinates ( [symbol] , [symbol] ). With the anchor and retinal coordinates defined, the actual biomarker prediction can proceed. To identify biomarkers, segmentation and quantification of the image is done for each specific biomarker. This task can be separated into two modules for segmentation and quantification respectively, or they can be combined into one biomarker identification module. As an example, the following disclosure explains how two separate learning modules are constructed and trained. Drusen is used as an example, but other biomarkers will adopt a similar learning process.

To achieve uncompromised segmentation accuracy on full resolution MSI images while maintaining global context, a custom dual-encoder modification of U-Net++ with EfficientNetV2 L-size encoder and multi-scale feature fusion module that accepts both local and global patches is used for the purpose of 2.5D binary semantic segmentation of drusen and other biomarkers. This scheme allows the model to produce high resolution segmentations of local patches while utilizing the global context to correctly treat boundary regions and more accurately discern drusen from similar looking lesions according to the location in fundus image.

For model training, a diverse MSI dataset covering a wide range of cases for a particular retinal disease such as AMD and adversarial cases featuring visually similar but distinct pathologies was composed. The model was trained, validated and evaluated on a large dataset with both positive and adversarial cases, respectively. All sets were obtained by stratified sampling with respect to the subcategories. Both validation and evaluation sets were balanced. Data augmentation can also include affine spatial transformation and intensity transformations.

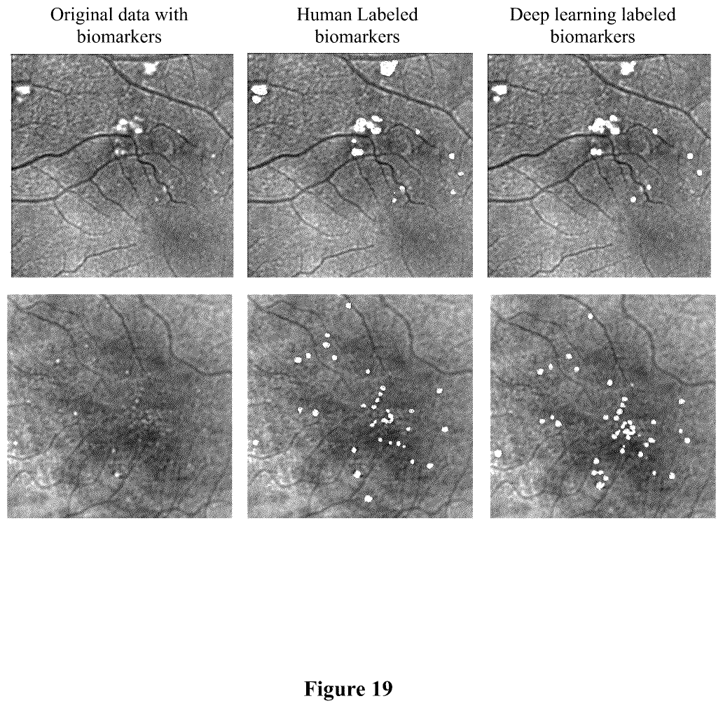

FIG. 19 provides a drusen segmentation model qualitative results with images of drusen identified by human and by the present method. The comparison of ground truth segmentation masks was provided by a specialist clinician with the predictions obtained from the final drusen segmentation model. A high degree of overlap is observed between the two, indicating the strong predictive capabilities of the model.

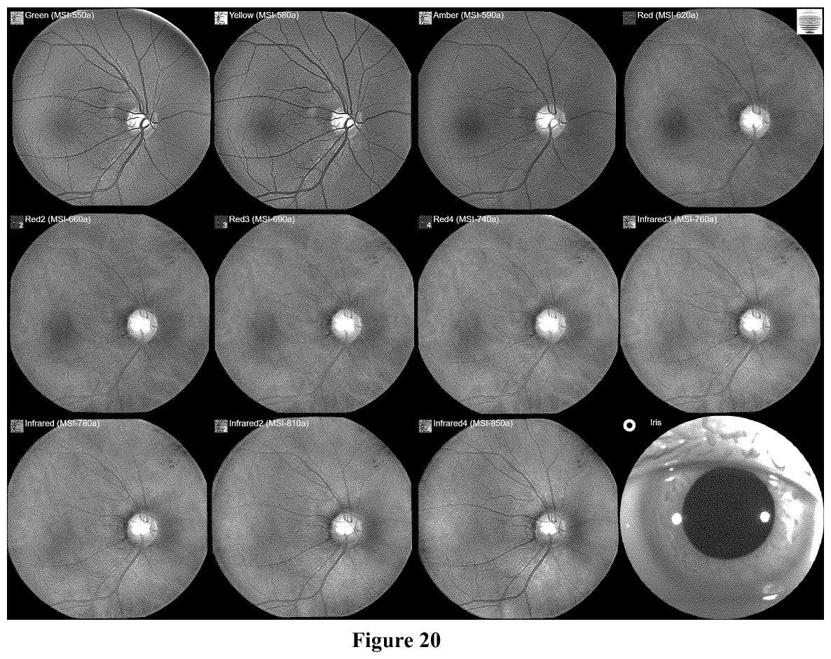

FIG. 20 is an example of a set of multi-spectral retinal images with visible biomarkers (drusen) appearing only with wavelengths >660 nm. Drusen are yellow deposits under the retina made up of lipids and proteins. Drusen can be different sizes and small drusen are common in people aged fifty and older without age-related macular degeneration (AMD). However, the presence of many small drusen and larger drusen are often signs of AMD.

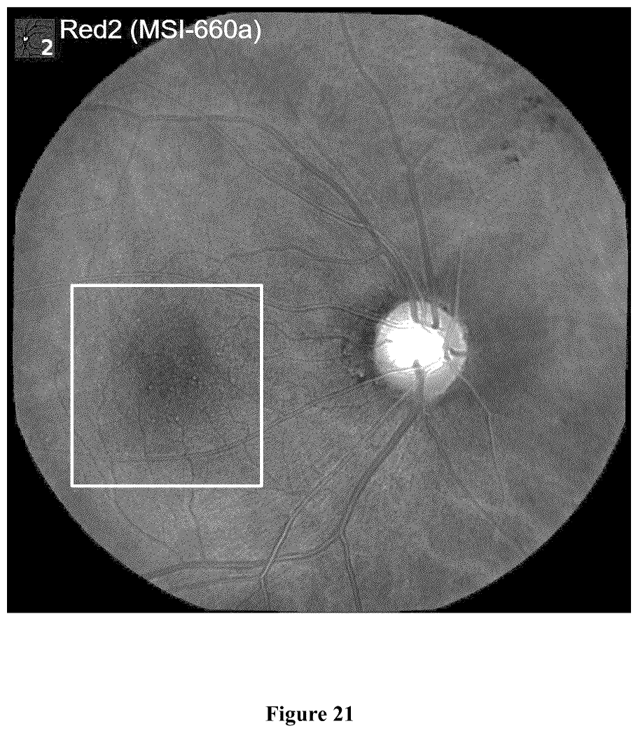

FIG. 21 is a close-up image at 660 nm showing drusen not visible in images at other wavelengths. As visible in the box, drusen present at 660 nm as bright spots of varying size, and are not present at other imaging wavelengths.

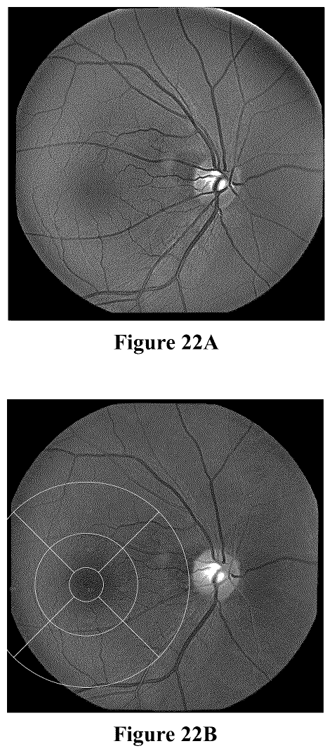

FIG. 22A is an example of an MSI image that does not have identified biomarkers, and FIG. 22B is an example of an MSI image that has identified and highlighted biomarkers. The resulting deep learning analysis of all MSI wavelengths has resulted in a map of biomarker presence and type at multiple wavelengths. The present method can identify that drusen are viewable in images present in a particular range of wavelengths at a particular location on the macula. In this case drusen can be automatically identified, quantified, and registered resulting drusen data is overlaid on top of the original image data to assist in rapid diagnosis.

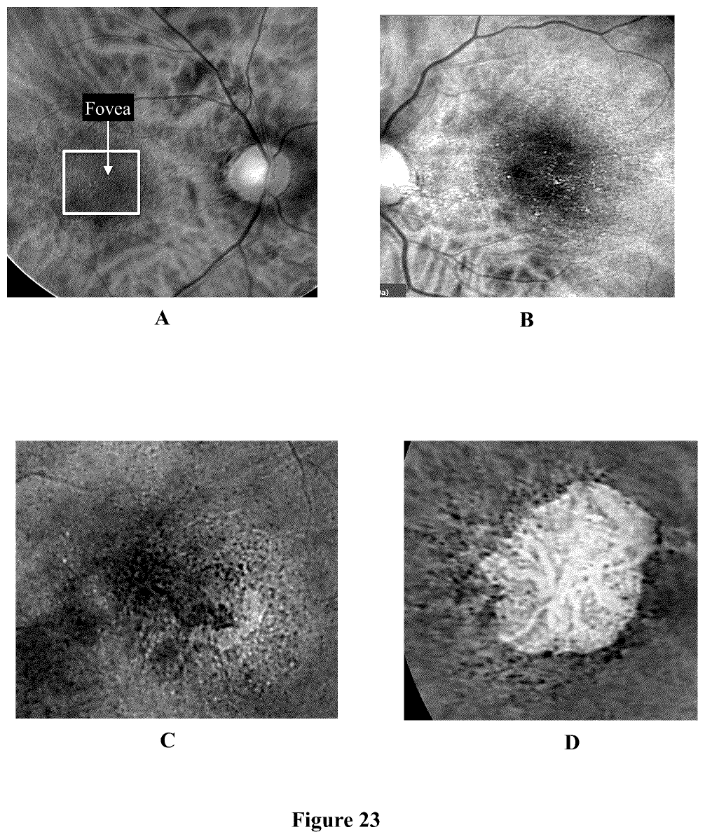

FIG. 23 illustrates identification of biomarkers in multi-spectral retinal images. MSI imaging technology with a selective range of wavelengths can be used to support early detection and rate of progression of Dry AMD. This can be observed from early to mild Retinal Pigmented Epithelium (RPE) depigmentation followed by increased pigmentation to focal atrophic areas and then large areas of geographical atrophy (GA), or lesions surrounded with pigmentation. Direct observation of hyperpigmentation or perilesional melanin clustering, in MSI imaging is a promising biomarker for assessment of disease progression towards GA. Image A illustrates focal depigmentation. Image B illustrates small drusen and hyperpigmentation, or areas of pigment clumping. Image C illustrates hyperpigmentation cluster with small drusen and atrophic area or RPE. Image D illustrates an area of hyperpigmentation on the boundary of a large GA. Further, temporal data supports determining direction and rate of progression of retinal disease and MSI with image processing as presently described can be used to monitor disease progression.

The present method can be used for any retinal biomarker identification and for a range of ocular biomarkers identification, extraction, and quantification from multi-spectral retinal images. The example provided herein is for drusen, however the method has been and can be applied to other biomarkers including DRY AMD biomarkers such as increased RPE pigmentation (melanin) related to AMD, RPE depigmentation areas, geographic atrophy, cardiovascular disease, among others. In addition, the present training method can be used with minimum adaptation for AI deployment in clinical usage. Furthermore, in clinical use, the results generated can be used to interact with clinicians and experts to provide feedback on results presented by deep learning. At every location where instruments are deployed, during review of MSI images and AI data, clinicians can indicate if there are false positives, false negatives, or an expert can accept the results related to specific biomarkers. This allows the resulting feedback to advance further training of the AI model, enabling continuous learning and accelerated development of specific disease biomarkers.

All publications, patents and patent applications mentioned in this specification are indicative of the level of skill of those skilled in the art to which this invention pertains and are herein incorporated by reference. The reference to any prior art in this specification is not, and should not be taken as, an acknowledgement or any form of suggestion that such prior art forms part of the common general knowledge.

The invention being thus described, it will be obvious that the same may be varied in many ways. Such variations are not to be regarded as a departure from the scope of the invention, and all such modifications as would be obvious to one skilled in the art are intended to be included within the scope of the following claims.

We claim:

1. A method of ocular biomarker identification comprising:
obtaining a plurality of digital multi-spectral images of a retina of a patient at a plurality of illumination wavelengths using a multi-spectral ophthalmoscope;
registering the plurality of multi-spectral images to provide a plurality of processed multi-spectral images that are scaled and aligned;
identifying an ocular biomarker in at least one of the plurality of multi-spectral images; and
automatically quantifying the biomarker based on the location and size of the biomarker in the plurality of processed multi-spectral images and the illumination wavelength at which the biomarker was identified.

2. The method of claim 1, wherein identifying the biomarker is based on the quantification of the biomarker at specific illumination wavelengths.

3. The method of claim 1, wherein the biomarker is one or more of a dry age-related macular degeneration (AMD) biomarker, diabetic retinopathy (DR) biomarker, geographic Atrophy (GA) biomarker, retinal tear, retinal detachment, hypertensive retinopathy, sickle cell retinopathy biomarker, epiretinal membrane (ERM) biomarker, central retinal vein occlusion (CRVO), branch retinal vein occlusion (BRVO), macular hole, retinitis pigmentosa biomarker, glaucoma biomarker, Stargardt disease biomarker, and cardiovascular biomarker.

4. The method of claim 1, wherein presence of the biomarker at a specific illumination wavelength enables differentiation of ocular biomarkers associated with specific ophthalmic diseases.

5. The method of claim 1, wherein registering the plurality of multi-spectral images comprises: identifying one or more retinal anchor and anatomical landmark; and defining retinal geographical coordinates.

6. The method of claim 1, wherein quantifying the biomarker comprises measurement of one or more of biomarker size, shape, density, intensity variations across spectral bands, and morphological characteristics.

7. The method of claim 1, wherein each of the plurality of multi-spectral image is a wide field of view image.

8. The method of claim 1, wherein the plurality of illumination wavelengths are selected based on the patient eye pigmentation.

9. The method of claim 1, wherein assessing quality of each of the plurality of processed multi-spectral images is done using a convolutional neural network based image qualification model.

10. The method of claim 1, wherein the plurality of multi-spectral digital images are obtained at illumination wavelengths about every 30-50 nm in the wavelength range of about 450 nm to about 940 nm.

11. The method of claim 1, wherein the plurality of illumination wavelengths includes autofluorescence wavelengths and infrared wavelengths.

12. The method of claim 1, wherein registering the plurality of multi-spectral images comprises one or more of denoising, artifact removal, geometric correction, contrast enhancement, and illumination equalization.

13. The method of claim 1, wherein registering the plurality of multi-spectral images comprises one or more of aligning the plurality of multi-spectral images to a common coordinate system, identifying a fovea center, and identifying an optic disk.

14. The method of claim 1, wherein each of the plurality of multi-spectral digital images is obtained by a multi-spectral ophthalmoscope in about 10 to 250 milliseconds, and the plurality of multi-spectral digital images are obtained by the multi-spectral ophthalmoscope in less than one second.

15. A system for ocular biomarker identification comprising:
a multi-spectral ophthalmoscope to capture a plurality of multi-spectral ocular images at a plurality of illumination wavelengths;
an image registration module to pre-process and register the plurality of multi-spectral ocular images;
a biomarker extraction module for isolating and quantifying an ocular biomarker in the plurality of multi-spectral ocular images, the biomarker extraction module comprising:
a biomarker segmentation sub-module comprising a deep learning algorithm to differentiate relevant biomarkers from a background ocular structure; and
a biomarker quantization sub-module.

16. The system of claim 15, wherein the biomarker extraction module further comprises a quality assessment module and region of interest (ROI) extraction module.

17. The system of claim 15, wherein the multi-spectral ophthalmoscope comprises an illumination system capable of ocular illumination at a plurality of illumination wavelengths in the range of about 450 nm and 940 nm.

18. The system of claim 15, wherein the biomarker quantization sub-module can provide a measurement of one or more of biomarker size, shape, density, intensity variations across spectral bands, and morphological characteristics of biomarkers in the plurality of multi-spectral ocular images.

19. The system of claim 15, wherein the biomarker extraction module is trained to identify an ocular biomarker selected from the group consisting of an dry age-related macular degeneration (AMD) biomarker, diabetic retinopathy (DR) biomarker, geographic Atrophy (GA) biomarker, retinal tear, retinal detachment, hypertensive retinopathy, sickle cell retinopathy biomarker, epiretinal membrane (ERM) biomarker, central retinal vein occlusion (CRVO), branch retinal vein occlusion (BRVO), macular hole, retinitis pigmentosa biomarker, glaucoma biomarker, Stargardt disease biomarker, and cardiovascular biomarker.

20. The system of claim 15, wherein the image registration module comprises a convolutional neural network to scale, align, and apply geographical coordinates to the plurality of multi-spectral images.
